SEQUENCE LISTING (5 sequences)

SEQ ID NO: 1 - protein, 972 aa, Artificial Sequence
  Human CSF-1R sequence
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 2 - protein, 277 aa, Artificial Sequence
  Amino acid sequence for CSF-1R
IPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVV

SEQ ID NO: 3 - protein, 49 aa, Artificial Sequence
  Amino acid sequence for CSF-1R (ENSP00000421174 49 amino acids)
MRHTNYSFSPWHGFTIHRAKFIQSQDYQCSALMGGRKVMSISIRLKVQK

SEQ ID NO: 4 - protein, 493 aa, Artificial Sequence
  Amino acid sequence for CSF-1R (SNP V32G, A245S, H247P, V279M, position underlined)
IPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIHQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDE

SEQ ID NO: 5 - protein, 512 aa, Artificial Sequence
  CSF-1R form suitable for expression on a cell surface
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDE

(19) United States
(12) Patent Application Publication
(10) Pub. No.: US 2016/0200820 A1
(43) Pub. Date: Jul. 14, 2016

(54) METHOD FOR THE TREATMENT OF FIBROTIC DISEASE

(71) Applicant: UCB BIOPHARMA SPRL, Brussels (BE)

(72) Inventor: Diane MARSHALL, SLOUGH, BERKSHIRE (GB)

(21) Appl. No.: 14/914,673

(22) PCT Filed: Aug. 26, 2014
(86) PCT No.: PCT/EP2014/068047

§ 371 Date: Feb. 26, 2016

(30) Foreign Application Priority Data
Aug. 30, 2013 (GB) 1315486.9

(51) Int. Cl. C07K 16/28; A61K 39/395; A61K 45/06; C12N 15/113

(52) U.S. Cl. CPC C07K 16/2866; C12N 15/1138; A61K 39/3955; A61K 45/06; C07K 2317/76; C07K 2317/92; C12N 2320/30

(57) ABSTRACT

The present invention relates generally to methods of treating fibrotic disease and more specifically to an inhibitor of CSF-1R activity for the treatment and/or prophylaxis of fibrotic disease, wherein the inhibitor comprises a small chemical entity (NCE), a nucleic acid or an antibody or functionally active fragment or derivative thereof. The present invention further pertains to the use of an inhibitor of CSF-1R activity, for the manufacture of a medicament for the treatment and/or prophylaxis of fibrotic disease.

The present invention relates generally to methods of treating fibrotic disease and more specifically to an anti-CSF1-R antibody for the treatment of fibrotic disease.

Colony stimulating factor 1 (CSF-1), also known as macrophage colony stimulating factor (M-CSF) is a cytokine produced by a variety of cells, including endothelial cells and fibroblasts. CSF-1 is composed of two “monomer” polypeptides, which form a biologically active dimeric CSF-1 protein. CSF-1 exists in at least three mature forms due to alternative RNA splicing, proteolytic processing of protein precursors and post-translational modifications including glycosylation and addition of proteoglycan (see, Cerretti D P et al. 1988, Mol Immunol, 25(8), 761; Pixley F J and Stanley E R, 2004, Trends in Cell Biology, 14(11) 628-38; Douglass, T G et al, 2008, Int Immunopharmacol, 8, 1354-76). The variousforms of CSF-1 protein include two secreted molecules, one that is glycosylated, the other comprised of a longer amino terminal sequence and proteoglycan modification. Another variant is a transmembrane (TM) molecule that is glycosylated but has no proteoglycan moieties. This membrane form can be shed via proteolytic cleavage to release an active, soluble molecule. All forms are produced as precursor polypeptides having a 32 amino acid signal sequence at the amino terminus, a putative transmembrane region of approximately 23 amino acids near the carboxyl terminus and a short cytoplasmic COOH-terminal tail. The precursor peptides are subsequently processed by amino terminal and carboxyl terminal proteolytic cleavages to produce the mature forms of CSF-1 with residues 1-149 being identical and constituting the receptor binding domain. In vivo, CSF-1 monomers are glycosylated, and dimerized via disulfide-linkage. CSF-1 belongs to a group of biological agonists that promote the production of blood cells. Specifically, it acts as a growth, differentiation and survival factor for bone marrow progenitor cells of the mononuclear phagocyte lineage. Further, CSF-1 stimulates the survival, proliferation and function of macrophages via a specific receptor on responding cells.

The CSF-1 receptor (CSF-1R) is also referred to as the c-fms gene product or CD115. CSF-1R is a 165 kDa type 1 TM glycoprotein belonging to the type III receptor tyrosine kinase family. In addition to CSF-1, the structurally similar but sequence unrelated molecule IL-34 has also been shown to be a ligand for CSF-1R (Lin, et al. 2008, Science 320:807-811). Expression of CSF-1R is restricted mainly to cells of the monocyte-macrophage lineage, both circulating and resident tissue populations, including osteoclasts. In addition, it is expressed in a number of cells of the female reproductive system including oocytes, decidual cells and trophoblasts (Pollard J W and Stanley E R, 1996 Advances in Developmental Biochemistry Vol 4, 1996, Pages 153-193 (Pleiotropic Roles for CSF-1 in Development Defined by the Mouse Mutation Osteopetrotic); Arceci R J, PNAS 1989, 86(22), 8818-8822 (Temporal Expression and Location of CSF-1 and its Receptor in Female Reproductive Tract are consistent with CSF-1-Regulated Placental Development); Arceci, R J et al, 1992, 151 (1), 1-8; Dev Biol; Regenstreif L J and Rossant J, Dev Biol 1989 May; 133(1): 284-94 (Expression of the c-fms-oncogene and of the cytokine, CSF-1, during mouse embryogenesis), Pampfer S et al, Biol Reprod 1992, 46(1), 48-57 (Expression of the CSF-1 receptor (c-fms proto-oncogene product) in the human uterus and placenta; Jokhi P P et al, Lab Invest 1993, 68(3), 308-320 (Expression of the CSF-1 Receptor (c-fms product) by cells at the human uteroplacental interface); Kauma S W et al, J Clin Endocrinol Metab 1991, 73(4), 746-751 (CSF-1 and c-fms expression in human endometrial tissues and placenta during the menstrual cycle and early pregnancy), Byrne J Cell Biol 1981 91(3 Pt 1) 848-53, Hofstetter W et al, Bone 1995, 17, (2), 145-151; Tanaka S et al, 1993, J Clin Invest, 91: 257-63; Weir E C et al, 1993, J Bone Miner Res, 8(12) 1507-18.

Binding of the ligand CSF-1 to the CSF-1 receptor results in the phosphorylation of the receptor on one or more tyrosine residues through the action of its tyrosine kinase domain. This phosphorylation can be detected because antibodies are available that bind to the receptor only after phosphorylation (for example Phospho-M-CSF-Receptor (Tyr546) antibody #3083 from Cell Signaling Technology).

Antibodies to CSF-1R are known in the art. Sherr, C. J. et al., Blood 73 (1989) 1786-1793 describes antibodies against CSF-1 R that inhibit the CSF-1 activity (Sherr, C. J. et al., Blood 73 (1989) 1786-1793). WO09/026303 discloses anti-CSF-1R antibodies which bind to human CSF-1R and in vivo mouse tumour models using an anti-murine CSF-1R antibody. WO11/123381 discloses anti-CSF-1R antibodies which internalize CSF-1R and have ADCC activity. WO11/123381 discloses in vivo mouse tumour models using an anti-murine CSF-1R antibody. WO11/140249 discloses anti-CSF-1R antibodies which block binding of CSF-1 to CSF-1R which are stated to be useful in the treatment of cancer. WO09/112245 discloses an anti-CSF-1R IgG1 antibody which inhibits CSF-1 binding to CSF-1R and is stated to be useful in the treatment of cancer, inflammatory bowel disease and rheumatoid arthritis. WO11/131407 discloses an anti-CSF-1R antibody which inhibits CSF-1 binding to CSF-1R and is stated to be useful in the treatment of bone loss and cancer. WO11/107553 discloses an anti-CSF-1R antibody which inhibits CSF-1 binding to CSF-1R and is stated to be useful in the treatment of bone loss and cancer. WO11/070024 discloses anti-CSF-1R antibodies which bind to human CSF-1R fragment delD4.

Anti-CSF-1R antibodies are currently in development for treatment of cancer and Rheumatoid Arthritis.

The term ‘fibrotic disease’ refers to an aberrant wound healing response wherein excess fibrous connective tissue is formed in an organ or tissue. The deposition and accumulation of excess extracellular matrix (ECM) components such as collagen and fibronectin, results in the hardening and scarring of tissues that ultimately can lead to organ failure.

Wounding results in an immediate coagulation and clotting response with the development of a provisional extracellular matrix (ECM). The initial platelet aggregation and activation helps promote an inflammatory response characterised by vasodilation and an increase in blood vessel permeability, allowing the recruitment of a variety of immune cells including neutrophils, macrophages, eosinophils and lymphocytes to the wound site. Neutrophils and macrophages debride the wound thereby reducing the risk of infection and together with activated lymphocytes secrete a variety of growth factors and cytokines that serve to further amplify the inflammatory response. Molecules such as TGFβ, PDGF and IL-13 activate macrophages and lead to the recruitment, proliferation and activation of fibroblasts at the wound site. Activated fibroblasts or myofibroblasts, are characterised by the expression of α-smooth muscle actin and secrete collagen and other ECM components. The activated fibroblasts, contract the collagen lattice, drawing the edges of the wound to the centre. Epithelial and endothelial cells proliferate and migrate over the temporary matrix to regenerate the damaged tissue which is coupled with resolution of the matrix components, completing the wound repair.

However, persistent tissue insult or injury, or a disregulation of the repair pathway leads to an inappropriate wound response. Excess deposition and hyper cross-linking of the collagen and ECM then occurs, resulting in excessive formation and hardening of scar tissue in place of the normal tissue architecture.

A number of approaches have shown that disease severity is reduced when macrophages are depleted in mouse models of lung fibrosis (Zhang-Hoover J et al, Immunology, 2000, 101(4): 501-511; Gibbons M A et al, Am J Respir Crit Care Med, 2011, 184(5); Murray, L A et al, 2011, Int J Biochem Cell Biol, 43, 154-62). Depletion was achieved either through administration of liposomal clodronate or use of a CD11c-diphtheria toxin receptor transgenic mouse, where monocyte/macrophage cells were ablated via injection of the diphtheria toxin molecule. Similarly, in a model of liver fibrosis (Duffield J S et al, J Clin Invest, 2005, 115(1): 56-65) and renal fibrosis (Lin S L, et al, 2009, J Immunol, 183, 6733-43), macrophage depletion reduced pathological damage and collagen deposition. The op/op mouse has a mutation in the CSF-1 ligand DNA sequence that results in the expression of a truncated, non-functional protein. These mice manifest an osteopetrotic phenotype and are deficient in macrophages. When utilised in a bleomycin model of lung fibrosis, disease severity is greatly reduced compared to wild type mice (Baran et al, 2007, Am J Respir Crit Care Med, 176, 78-89).

Complexity is added to the role of macrophages as they can be classified into at least two functional phenotypes, M1 and M2 (Mantovani, A et al, 2004, Trends in Immunol, 25(12), 677-86; Mantovani, A et al, 2007, Eur J Immunol, 37, 14-16; Mosser D M and Edwards J P, 2008, Nat Rev Immunol, 8, 958-) where M2 macrophages possess pro-fibrotic characteristics (Lupher, M L and Gallatin, W M, 2006, Adv Immunol, 89, 245-88; Prasse A et al, 2006, Am J Resp Crit Care Med, 173, 781-92; Pechkovsky, D V et al, 2010, Clin Immunol, 137 (1), 89-1-1; Gibbons M A et al, 2011, Am J Resp Crit Care Med, 184, 569-81).

Examples of fibrotic diseases include pulmonary fibrosis, such as idiopathic pulmonary fibrosis (IPF), silicosis, cystic fibrosis, renal fibrosis, liver fibrosis, liver cirrhosis, primary sclerosing cholangitis, primary biliary cirrhosis, endomyocardial fibrosis, mediastinal fibrosis, myelofibrosis, retroperitoneal fibrosis, progressive massive fibrosis, nephrogenic systemic fibrosis, Crohn's disease, keloid, myocardial infarction, systemic sclerosis and arthrofibrosis.

The cause of fibrotic disease can be dependent upon the organ or tissue involved and is unknown in some diseases such as idiopathic pulmonary fibrosis. Liver fibrosis and ultimately cirrhosis results from chronic liver damage sustained through exposure to a variety of factors including environmental and dietary factors or infectious agents. Long-term hepatitis B or C infections can cause liver fibrosis. Sustained over consumption of alcohol or a high fat/sugar diet can also lead to cirrhosis of the liver. Similarly, diabetes can damage and scar the kidneys leading to loss of function. In familial pulmonary fibrosis, family members have an increased susceptibility to fibrosis although the genetic factor(s) responsible are unknown.

IPF is a chronic, progressive disease, of unknown cause and is one of seven idiopathic interstitial pneumonias (American Thoracic Society/European Respiratory Society Multidisciplinary Consensus Classification of the Idiopathic Interstitial Pneumonias, 2002, Am J Respir Crit Care Med, 165:277-304). Repetitive injury to alveolar epithelial cells is believed to play a key role in promoting the fibrotic process that results in the typical histopathological features of IPF (Selman M, 2001, Ann Intern Med, 134(2): 136-51; Gross T J and Hunninghake G W, 2001). A number of risk factors may cause the epithelial cell injury, particularly inhaled toxins and agents. Smoking has been identified as one such likely factor while exposure to wood dusts, metal dusts, silica or other mineral dusts have been reported to increase the risk of developing IPF (reviewed in Borchers A T et al, 2011 Clinic Rev Allerg Immunol, 40: 117-134); Maher T M, 2012, Clin Chest Med, 33: 69-83). The prognosis for patients with IPF is poor with the median survival range being 2-5 years following diagnosis (Gribbin J, et al, 2006, Thorax, 61: 980-85; King T E Jr, 2001, Am J Respir Care Med, 1664: 1171-81; Fernandez Perez E R et al, 2010, Chest, 137: 129-37; Lee H L et al, 2005, Chest, 127:2034-41).

Treatment of fibrotic disease typically includes anti-inflammatory and immunosuppressive agents but these are of little benefit for the patient. The lack of efficacy with these treatments contributed to the reconsideration of IPF and fibrotic disease in general, as an aberrant response to wound healing and not an inflammatory condition. Pirfenidone is a small molecule drug that was approved for use in the treatment of IPF in Japan in 2008 and Europe in 2011 which is likely to work via multiple mechanisms of action that are not completely understood but may work in part through a down-regulation of TGF-β. To date, no targeted therapies have been approved for fibrotic indications.

Therefore, there is currently an unmet medical need for improved treatment of fibrotic disease. For example, for IPF there is a 3 year survival rate of 50% and a 5 year survival rate of only 20% and transplantation is required in about 20% of cases.

Accordingly, it is an object of the present invention to provide a new method of treatment of fibrotic disease.

### SUMMARY OF THE DISCLOSURE

Surprisingly we have been able to demonstrate that inhibitors of CSF-1R activity are active in the treatment of fibrotic disease. Specifically, we have been able to demonstrate that an anti-CSF-1R antibody that inhibits CSF-1R activity is active in in vivo animal models of pulmonary fibrosis.

Accordingly, the present invention provides an inhibitor of CSF-1R activity, for the treatment and/or prophylaxis of fibrotic disease. The invention also provides the use of an inhibitor of CSF-1R activity, for the manufacture of a medicament for the treatment and or prophylaxis of fibrotic disease. The invention further provides a method for the treatment and/or prophylaxis of fibrotic disease comprising administering a therapeutically effective amount of an inhibitor of CSF-1R activity.

In the present application, the term “fibrotic disease” includes diseases that are characterised by an aberrant response to wound healing wherein excess fibrous connective tissue is formed in an organ or tissue. Illustrative fibrotic diseases include but are not limited to include pulmonary fibrosis, such as idiopathic pulmonary fibrosis, silicosis and cystic fibrosis; renal fibrosis including tubular atrophy and interstitial fibrosis, liver fibrosis and liver cirrhosis, primary sclerosing cholangitis, primary biliary cirrhosis, endomyocardial fibrosis, mediastinal fibrosis, myelofibrosis, retroperitoneal fibrosis, progressive massive fibrosis, nephrogenic systemic fibrosis, Crohn's disease, keloid, myocardial infarction, scleroderma, systemic sclerosis and arthrofibrosis.

The term ‘CSF-1R activity’ as used herein refers to the spectrum of activity understood in the art for CSF-1R, in particular the activity of human CSF-1R and isoforms thereof, for example 1, 2, 3 or all isoforms. For example, binding of ligand to the receptor induces phosphorylation of CSF-1R at specific tyrosine residues (Bourette R P and Rohrschneider L R, 2000, Growth Factors 17: 155-166) and the ensuing cascade of signal transduction events can mediate cell migration, survival, differentiation and proliferation (Suzu S et al, 1997, J Immunol, 159, 1860-7; Yeung Y-G and Stanley E R, 2003, Mol Cell Proteomics, 2, 1143-55; Yu W et al 2008, J Leukoc Biol 84(3), 852-63). Expression in transfected cells of mutant CSF-1R receptor molecules comprising phenylalanine residues in place of selected tyrosine residues revealed the association of specific tyrosine residues with cellular outcomes such as survival, proliferation and morphology (Yu et al J Leukoc Biol 2008 September 84(3): 852-863). Proteomic approaches and immunoblotting techniques using anti-phosphotyrosine antibodies together with molecule specific antibodies, have identified a number of the intracellular molecules involved in mediating these cell functions following ligand stimulation of the receptor (Yeung Y-G et al, 1998, J Biol Chem. 13, 273(46): 17128-37; Husson H et al, 1997, Oncogene 15, 14(19): 2331-8.

In one embodiment the subject is a mammal, for example a human.

An inhibitor of CSF-1R activity according to the present invention is an agent that interferes with, for example reduces/inhibits or blocks the activity of CSF-1R, in particular the activity of CSF-1R in fibrotic disease. Particularly preferred are agents which interfere with the activity of CSF-1R in IPF. Inhibitors according to the present invention may partially or completely inhibit CSF-1R activity Inhibitors of use in the present invention include without limitation, inhibitors that are capable of interacting with (e.g. binding to, or recognising) IL-34, CSF-1 or the CSF-1 receptor (CSF-1R) or a nucleic acid molecule encoding IL-34, CSF-1 or CSF-1R, or are capable of inhibiting the expression of IL-34, CSF-1 or CSF-1 R or are capable of inhibiting the interaction between CSF-1R and CSF-1 and/or IL-34. Such inhibitors may be, without limitation, antibodies, nucleic acids (e.g. DNA, RNA, antisense RNA and siRNA), carbohydrates, lipids, proteins, polypeptides, peptides, peptidomimetics, small molecules and other drugs. In one embodiment the inhibitor blocks binding of CSF-1 to the receptor CSF-1R.

Examples, of suitable inhibitors include, but are not limited to, a synthetic functional fragment of the CSF-1 receptor that binds to CSF-1 and interferes with binding to the native CSF-1 receptor, a synthetic functional fragment of CSF-1 that binds to CSF-1 receptor and interferes with binding to the native CSF-1 receptor, a synthetic functional fragment of IL-34 that binds to CSF-1 receptor and interferes with binding to the native CSF-1 receptor, an antibody that binds to CSF-1 or IL-34 or to the CSF-1 receptor and interferes with CSF-1 receptor-ligand interaction, an antisense nucleic acid molecule that specifically hybridizes to mRNA encoding CSF-1, IL-34 or the CSF-1 receptor or a small molecule or other drug which inhibits the activity of IL-34, CSF-1 or CSF-1R.

Inhibitors of CSF-1 receptor activity are known in the art as are methods of identifying and producing such inhibitors. Neutralising anti-CSF-1 antibodies have been described, for example by Weir et al., 1996, J Bone Miner. Res.l 1, 1474-1481 and Haran-Ghera et al, 1997, Blood, 89, 2537-2545, which also describes anti-CSF-1R antibodies. Antisense antagonists of CSF-1 have also been described (EP1223980).

Agents that may be suitable inhibitors can be selected from a wide variety of candidate agents. Examples of candidate agents include but are not limited to, nucleic acids (e.g. DNA and RNA), carbohydrates, lipids, proteins, polypeptides, peptides, peptidomimetics, small molecules and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is suited to peptide libraries, while the other four approaches are applicable to peptide, non-peptide oligomer or small molecule libraries of compounds (Lam, 1997, Anticancer Drug Des. 12:145; U.S. Pat. No. 5,738,996; and U.S. Pat. No. 5,807,683).

Examples of suitable methods based on the present description for the synthesis of molecular libraries can be found in the art, for example in: DeWitt et al, 1993, Proc. Natl. Acad. Sci. USA 90:6909; Erb et al, 1994, Proc. Natl. Acad. Sci. USA 91:11422; Zuckermann et al, 1994, J. Med. Chem. 37:2678; Cho et al, 1993, Science 261:1303; Carrell et al, 1994, Angew. Chem. hit. Ed. Engl. 33:2059; Carell et al, 1994, Angew. Chem. Int. Ed. Engl. 33:2061; and Gallop et al, 1994, J. Med. Chem. 37:1233.

Libraries of compounds maybe presented, for example, in solution (e.g. Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al, 1992, Proc. Natl. Acad. Sci. USA 89:1865-1869) or phage (Scott and Smith, 1990, Science 249:386-390; Devlin, 1990, Science 249:404-406; Cwirla et al, 1990, Proc. Natl. Acad. Sci. USA 87:6378-6382; and Felici, 1991, J. Mol. Biol. 222:301-310).

In one example, the inhibitor for use in the present invention may be a nucleic acid. In particular CSF-1, IL-34 or CSF-1R nucleic acid molecules may be used as anti-sense molecules, to alter the expression of their respective polypeptides by binding to complementary nucleic acids. CSF-1, IL-34 or CSF-1R nucleic acids may be obtained using standard cloning techniques from for example genomic DNA or cDNA or can be synthesised using well known and commercially available techniques. The CSF-1, IL-34 or CSF-1R nucleic acids may contain one or more nucleotide substitutions, additions or deletions into the nucleotide sequence of a CSF-1, IL-34 or CSF-1 R nucleic acid. Standard techniques known to those of skill in the art can be used to introduce mutations, including, for example, site-directed mutagenesis and PCR-mediated mutagenesis. An antisense nucleic acid according to the present invention includes a CSF-1, IL-34 or CSF-1R nucleic acid capable of hybridising by virtue of some sequence complementarity to a portion of an RNA (preferably mRNA) encoding the respective polypeptide. The antisense nucleic acid can be complementary to a coding and/or non-coding region of an mRNA encoding such a polypeptide. Most preferably, the antisense nucleic acids result in inhibition of the expression of the CSF-1, IL-34 or CSF-1R polypeptide. Thus, the present invention provides a method for the treatment and/or prophylaxis of fibrotic disease comprising administering a therapeutically effective amount of an inhibitor of CSF-1R activity wherein the inhibitor comprises at least eight nucleotides (for example 15 to 22 nucleotides, such as 15, 16, 17, 18, 19, 20, 21 or 22 nucleotides) that are antisense to a gene or cDNA encoding a CSF-1, IL-34 or CSF-1R polypeptide. The invention also provides the use of nucleic acids comprising at least eight nucleotides (for example 15 to 22 nucleotides, such as 15, 16, 17, 18, 19, 20, 21 or 22 nucleotides) that are antisense to a gene or cDNA encoding a CSF-1, IL-34 or CSF-1R polypeptide for the manufacture of a medicament for use in the treatment and/or prophylaxis of fibrotic disease.

In one embodiment, the inhibitor for use in the treatment and/or prophylaxis of fibrotic disease is an antibody that inhibits the activity of CSF-1R. Accordingly, there is provided the use of an antibody that inhibits the activity of CSF-1R for the manufacture of a medicament for use in the treatment and/or prophylaxis of fibrotic disease. Also provided is a method of treatment and or prophylaxis of fibrotic disease in a subject comprising administering to said subject a therapeutically effective amount of an antibody that inhibits the activity of CSF-1R. In one embodiment, the inhibitor for use in the treatment and/or prophylaxis of fibrotic disease is an antibody that interacts with (i.e. binds to or recognises) CSF-1 or IL-34. In one example the antibodies selectively interact with CSF-1 or IL-34. Selectively interacting with (e.g. recognising or binding to) means that the antibodies have a greater affinity for CSF-1 or IL-34 polypeptides than for other polypeptides. Examples of suitable antibodies are those that inhibit the activity of CSF-1 or IL-34 by binding to CSF-1 or IL-34 in such a manner as to prevent it being biologically active, for example by preventing the binding of CSF-1 or IL-34 to its receptor. Accordingly, there is provided by the present invention the use of an anti-CSF-1 antibody or an anti-IL-34 antibody for the manufacture of a medicament for use in the treatment and/or prophylaxis of fibrotic disease.

Most preferably, an inhibitor for use in the treatment and/or prophylaxis of fibrotic disease is an antibody that interacts with (i.e. binds to or recognises) CSF-1R and inhibits the activity of CSF-1R. In one example the antibodies selectively interact with CSF-1R. Selectively interacting with (e.g. recognising or binding to) means that the antibodies have a greater affinity for CSF-1R polypeptide than for other polypeptides. Examples of suitable antibodies are those that inhibit the activity of CSF-1R by binding to CSF-1R in such a manner as to prevent it being biologically active.

In one embodiment the antibody recognises isoforms of CSF-1R, for example human CSF-1R and isoforms thereof.

In one embodiment the anti-CSF-1R antibody is capable of blocking ligand binding to CSF-1R. Blocking as employed herein refers to physically blocking such as occluding the receptor but will also include where the antibody or fragments binds an epitope that causes, for example a conformational change which means that the natural ligand to the receptor no longer binds. Assays suitable for determining the ability of an antibody to block CSF-1R are described in the Examples herein, see Example 2. BIAcore is an example of an assay employed to measure binding kinetics, ELISA assays or cell based assays employing monocytes or THP-1 cells may also be useful. CSF-1 and IL-34 are both ligands for CSF-1R and the anti-CSF-1R antibody may block binding of CSF-1 or IL-34 to CSF-1R but preferably blocks binding of both CSF-1 and IL-34 to CSF-1R. The anti-CSF-1R antibody also preferably does not cause CSF-1R activation or CSF-1R internalisation. Assays suitable for determining the ability of an antibody to cause CSF-1R activation or CSF-1R internalisation are described in the Examples, see Example 2 which describes an assay measuring CSF-1 dependent proliferation.

Accordingly, there is provided the use of an anti-CSF-1R antibody for the manufacture of a medicament for use in the treatment and/or prophylaxis of fibrotic disease. Also provided is a method of treatment and or prophylaxis of fibrotic disease in a subject comprising administering to said subject a therapeutically effective amount of an anti-CSF-1R antibody.

CSF-1, IL-34 or CSF-1R polypeptides or cells expressing said polypeptides can be used to produce antibodies which specifically recognise said polypeptides. The CSF-1, IL-34 and CSF-1 R polypeptides may be ‘mature’ polypeptides or biologically active fragments or derivatives thereof. Preferably the CSF-1 polypeptide contains amino acids 1-149 believed to be important for biological activity.

CSF-1R as employed herein refers to the protein named CSF-1R, isoforms thereof and biologically active fragments thereof. In one embodiment CSF-1R is human protein or an isoform thereof.

Generally an antibody employed in the present invention will be directed to the extracellular domain of CSF-1R.

FIG. 1A shows the full 972 amino acid sequence of human CSF1-R where residues 1-19 are the predicted signal peptide. Preferably the CSF-1R polypeptide contains amino acids 20-517 of the human CSF-1R sequence shown in FIG. 1A Amino acids 20-517 represent the predicted extracellular region of the CSF-1R sequence. FIG. 1B shows some alternative forms of CSF-1R.

Human CSF-1R is registered in the UniProt database under P07333.

CSF-1 and CSF-1 R polypeptides may be prepared by processes well known in the art from genetically engineered host cells comprising expression systems or they may be recovered from natural biological sources.

In one embodiment the sequence shown in FIG. 1C may be transfected into a suitable cell line and the polypeptide expressed on the cell surface. The amino acid fragment may be fused to a GPI-anchor to facilitate said expression. The cells may then be employed to immunize hosts.

In the present application, the term “polypeptides” includes peptides, polypeptides and proteins. These are used interchangeably unless otherwise specified. CSF-1 or CSF-1R polypeptides may in some instances be part of a larger protein such as a fusion protein for example fused to an affinity tag.

Antibodies generated against these polypeptides may be obtained by administering the polypeptides or cells expressing the same to an animal, preferably a non-human animal, using well-known and routine protocols, see for example Handbook of Experimental Immunology, D. M. Weir (ed.), Vol 4, Blackwell Scientific Publishers, Oxford, England, 1986. Many warm-blooded animals, such as rabbits, mice, rats, sheep, cows or pigs may be immunized. However, mice, rabbits, pigs and rats are generally preferred.

Monoclonal antibodies may be prepared by any method known in the art such as the hybridoma technique (Kohler and Milstein, 1975, Nature, 256:495-497), the trioma technique, the human B-cell hybridoma technique (Kozbor et al, 1983, Immunology Today, 4:72) and the EBV-hybridoma technique (Cole et al, Monoclonal Antibodies and Cancer Therapy, p 77-96, Alan R Liss, Inc., 1985).

Antibodies for use in the invention may also be generated using single lymphocyte antibody methods by cloning and expressing immunoglobulin variable region cDNAs generated from single lymphocytes selected for the production of specific antibodies by for example the methods described by Babcook, J. et al, 1996, Proc. Natl. Acad. Sci. USA 93(15):7843-7848, WO92/02551 and WO2004/051268 and WO2004/106377.

Specific as employed herein is intended to refer to an antibody that only recognises the antigen to which it is specific or an antibody that has significantly higher binding affinity to the antigen to which it is specific compared to binding to antigens to which it is non-specific, for example at least 5, 6, 7, 8, 9, 10 times higher binding affinity.

Chimeric antibodies are those antibodies encoded by immunoglobulin genes that have been genetically engineered so that the light and heavy chain genes are composed of immunoglobulin gene segments belonging to different species. These chimeric antibodies are likely to be less antigenic. Bivalent antibodies may be made by methods known in the art (Milstein et al, 1983, Nature 305:537-539; WO 93/08829, Traunecker et al, 1991, EMBO J. 10:3655-3659). Multi-valent antibodies may comprise multiple specificities or may be monospecific (see for example WO 92/22853).

In one embodiment the antibody for use in the present invention is humanised. As used herein, the term ‘humanised antibody molecule’ refers to an antibody molecule wherein the heavy and/or light chain contains one or more CDRs (including, if desired, one or more modified CDRs) from a donor antibody (e.g. a murine monoclonal antibody) grafted into a heavy and/or light chain variable region framework of an acceptor antibody (e.g. a human antibody) (see, e.g. U.S. Pat. No. 5,585,089; WO91/09967). For a review, see Vaughan et al, Nature Biotechnology, 16, 535-539, 1998.

In one embodiment rather than the entire CDR being transferred, only one or more of the specificity determining residues from any one of the CDRs described herein above are transferred to the human antibody framework (see for example, Kashmiri et al., 2005, Methods, 36, 25-34). In one embodiment only the specificity determining residues from one or more of the CDRs described herein above are transferred to the human antibody framework. In another embodiment only the specificity determining residues from each of the CDRs described herein above are transferred to the human antibody framework.

In a humanised antibody of the present invention, the framework regions need not have exactly the same sequence as those of the acceptor antibody. For instance, unusual residues may be changed to more frequently-occurring residues for that acceptor chain class or type. Alternatively, selected residues in the acceptor framework regions may be changed so that they correspond to the residue found at the same position in the donor antibody (see Reichmann et al., 1998, Nature, 332, 323-324). Such changes should be kept to the minimum necessary to recover the affinity of the donor antibody. A protocol for selecting residues in the acceptor framework regions which may need to be changed is set forth in WO91/09967.

When the CDRs or specificity determining residues are grafted, any appropriate acceptor variable region framework sequence may be used having regard to the class/type of the donor antibody from which the CDRs are derived, including mouse, primate and human framework regions.

Suitably, the humanised antibody has a variable domain comprising human acceptor framework regions as well as one or more of CDRs. Thus, provided in one embodiment is humanised antibody which binds human CSF-1, IL-34 or CSF-1R wherein the variable domain comprises human acceptor framework regions and non-human donor CDRs.

Examples of human frameworks which can be used in the present invention are KOL, NEWM, REI, EU, TUR, TEI, LAY and POM (Kabat et al., supra). For example, KOL and NEWM can be used for the heavy chain, REI can be used for the light chain and EU, LAY and POM can be used for both the heavy chain and the light chain. Alternatively, human germline sequences may be used; these are available at: http://vbase.mrc-cpe.cam.ac.uk/

In a humanised antibody of the present invention, the acceptor heavy and light chains do not necessarily need to be derived from the same antibody and may, if desired, comprise composite chains having framework regions derived from different chains.

The antibodies for use in the present invention can also be generated using various phage display methods known in the art and include those disclosed by Brinkman et al. (in J. Immunol. Methods, 1995, 182: 41-50), Ames et al. (J. Immunol. Methods, 1995, 184:177-186), Kettleborough et al (Eur. J. Immunol. 1994, 24:952-958), Persic et al. (Gene, 1997 187 9-18), Burton et al (Advances in Immunology, 1994, 57:191-280) and WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108. Techniques for the production of single chain antibodies, such as those described in U.S. Pat. No. 4,946,778 can also be adapted to produce single chain antibodies to CSF-1, IL-34 or CSF-1R polypeptides. Also, transgenic mice, or other organisms, including other mammals, may be used to express humanized antibodies.

The antibody used in the present invention may comprise a complete antibody molecule having full length heavy and light chains or a fragment thereof and may be, but are not limited to Fab, modified Fab, Fab′, modified Fab′, F(ab′)₂, Fv, single domain antibodies (e.g. VH or VL or VHH), scFv, bi, tri or tetra-valent antibodies, Bis-scFv, diabodies, triabodies, tetrabodies and epitope-binding fragments of any of the above (see for example Holliger and Hudson, 2005, Nature Biotech. 23(9):1126-1136; Adair and Lawson, 2005, Drug Design Reviews—Online 2(3), 209-217). The methods for creating and manufacturing these antibody fragments are well known in the art (see for example Verma et al., 1998, Journal of Immunological Methods, 216, 165-181). Other antibody fragments for use in the present invention include the Fab and Fab′ fragments described in International patent applications WO2005/003169, WO2005/003170 and WO2005/003171. Multi-valent antibodies may comprise multiple specificities e.g bispecific or may be monospecific (see for example WO 92/22853, WO05/113605, WO2009/040562 and WO2010/035012).

In one embodiment the antibody is provided as CSF-1, IL-34 or CSF-1R binding antibody fusion protein which comprises an immunoglobulin moiety, for example a Fab or Fab′ fragment, and one or two single domain antibodies (dAb) linked directly or indirectly thereto, for example as described in WO2009/040562, WO2010/035012, WO2011/030107, WO2011/061492 and WO2011/086091 all incorporated herein by reference.

In one embodiment the fusion protein comprises two domain antibodies, for example as a variable heavy (VH) and variable light (VL) pairing, optionally linked by a disulphide bond.

In one embodiment the Fab or Fab′ element of the fusion protein has the same or similar specificity to the single domain antibody or antibodies. In one embodiment the Fab or Fab′ has a different specificity to the single domain antibody or antibodies, that is to say the fusion protein is multivalent. In one embodiment a multivalent fusion protein according to the present invention has an albumin binding site, for example a VH/VL pair therein provides an albumin binding site.

Antibody fragments and methods of producing them are well known in the art, see for example Verma et al, 1998, Journal of Immunological Methods, 216, 165-181. Particular examples of antibody fragments for use in the present invention are Fab′ fragments which possess a native or a modified hinge region. A number of modified hinge regions have already been described, for example, in U.S. Pat. No. 5,677,425, WO99/15549, and WO98/25971 and these are incorporated herein by reference Further examples of particular antibody fragments for use in the present invention include those described in international patent applications PCT/GB2004/002810, PCT/GB2004/002870 and PCT/GB2004/002871 (all filed on 1 Jul. 2004). In particular the modified antibody Fab fragments described in International patent application PCT/GB2004/002810 are preferred.

In one embodiment the antibody heavy chain comprises a CH1 domain and the antibody light chain comprises a CL domain, either kappa or lambda.

In one embodiment the antibody heavy chain comprises a CH1 domain, a CH2 domain and a CH3 domain and the antibody light chain comprises a CL domain, either kappa or lambda.

The antibody can be of any class (e.g. IgG, IgE, IgM, IgD and IgA) or subclass of immunoglobulin molecule. In one embodiment the antibody for use in the present invention is of IgG class and may be selected from any of the IgG subclasses IgG1, IgG2, IgG3 or IgG4. Differential ADCC induction by different IgG isotypes is dependent on the affinity of these residues to FcγRs. In humans, IgG1 and IgG3 are known to induce effector functions whereas IgG2 and IgG4 induce effector functions weakly.

In a preferred embodiment the antibody for use in the present invention is an IgG2 or IgG4 antibody which induces effector functions weakly, including ADCC. It is particularly preferred for the antibody for use in treating fibrotic disease in the present invention to have limited effector function because the use of an antibody with effector function may cause enhanced depletion of cells expressing CSF-1R including macrophages which has potential to add to side effects in the patient.

The antibody for use in the present invention may include one or more mutations to alter the activity of the antibody. Angal et al (Angal, S., King, D. J., Bodmer, M. W., Turner, A., Lawson, A. D., Roberts, G., Pedley, B., and Adair, J. R. 1993. A single amino acid substitution abolishes the heterogeneity of chimeric mouse/human (IgG4) antibody. Mol. Immunol. 30:105-108) describes a site directed mutagenesis approach to minimize half-molecule formation of IgG4 antibodies. In this report, a single amino acid substitution within the core hinge, S241P, resulted in substantially less half-molecule formation. Accordingly, in the embodiment where the antibody is an IgG4 antibody, the antibody may include the mutation S241P.

It will also be understood by one skilled in the art that antibodies may undergo a variety of posttranslational modifications. The type and extent of these modifications often depends on the host cell line used to express the antibody as well as the culture conditions. Such modifications may include variations in glycosylation, methionine oxidation, diketopiperazine formation, aspartate isomerization and asparagine deamidation. A frequent modification is the loss of a carboxy-terminal basic residue (such as lysine or arginine) due to the action of carboxypeptidases (as described in Harris, R J. Journal of Chromatography 705:129-134, 1995). Accordingly, the C-terminal lysine of the antibody heavy chain may be absent.

This specific region or epitope of the human CSF-1, IL-34 or CSF-1R polypeptide can be identified by any suitable epitope mapping method known in the art in combination with any one of the antibodies provided by the present invention. Examples of such methods include screening peptides of varying lengths derived from CSF-1R for binding to the antibody of the present invention with the smallest fragment that can specifically bind to the antibody containing the sequence of the epitope recognised by the antibody. The CSF-1, IL-34 or CSF-1R peptides may be produced synthetically or by proteolytic digestion of the CSF-1, IL-34 or CSF-1R polypeptide. Peptides that bind the antibody can be identified by, for example, mass spectrometric analysis. In another example, NMR spectroscopy or X-ray crystallography can be used to identify the epitope bound by an antibody of the present invention. Once identified, the epitopic fragment which binds an antibody of the present invention can be used, if required, as an immunogen to obtain additional antibodies which bind the same epitope.

Biological molecules, such as antibodies or fragments, contain acidic and/or basic functional groups, thereby giving the molecule a net positive or negative charge. The amount of overall “observed” charge will depend on the absolute amino acid sequence of the entity, the local environment of the charged groups in the 3D structure and the environmental conditions of the molecule. The isoelectric point (pI) is the pH at which a particular molecule or solvent accessible surface thereof carries no net electrical charge. In one example, the CSF-1, IL-34 or CSF-1R antibody and fragments may be engineered to have an appropriate isoelectric point. This may lead to antibodies and/or fragments with more robust properties, in particular suitable solubility and/or stability profiles and/or improved purification characteristics.

Thus in one aspect the invention provides a humanised CSF-1, IL-34 or CSF-1R antibody engineered to have an isoelectric point different to that of the originally identified antibody. The antibody may, for example be engineered by replacing an amino acid residue such as replacing an acidic amino acid residue with one or more basic amino acid residues. Alternatively, basic amino acid residues may be introduced or acidic amino acid residues can be removed. Alternatively, if the molecule has an unacceptably high pI value acidic residues may be introduced to lower the pI, as required. It is important that when manipulating the pI care must be taken to retain the desirable activity of the antibody or fragment. Thus in one embodiment the engineered antibody or fragment has the same or substantially the same activity as the “unmodified” antibody or fragment.

Programs such as **ExPASY http://www.expasy.ch/tools/pi_tool.html, and

http://www.iut-arles.up.univ-mrs.fr/w3bb/d_abim/compo-p.html, may be used to predict the isoelectric point of the antibody or fragment.

It will be appreciated that the affinity of antibodies provided by the present invention may be altered using any suitable method known in the art. The present invention therefore also relates to variants of the antibody molecules of the present invention, which have an improved affinity for CSF-1, IL-34 or CSF-1R. Such variants can be obtained by a number of affinity maturation protocols including mutating the CDRs (Yang et al., J. Mol. Biol., 254, 392-403, 1995), chain shuffling (Marks et al., Bio/Technology, 10, 779-783, 1992), use of mutator strains of E. coli (Low et al., J. Mol. Biol., 250, 359-368, 1996), DNA shuffling (Patten et al., Curr. Opin. Biotechnol., 8, 724-733, 1997), phage display (Thompson et al., J. Mol. Biol., 256, 77-88, 1996) and sexual PCR (Crameri et al., Nature, 391, 288-291, 1998). Vaughan et al. (supra) discusses these methods of affinity maturation.

If desired an antibody for use in the present invention may be conjugated to one or more effector molecule(s). It will be appreciated that the effector molecule may comprise a single effector molecule or two or more such molecules so linked as to form a single moiety that can be attached to the antibodies of the present invention. Where it is desired to obtain an antibody fragment linked to an effector molecule, this may be prepared by standard chemical or recombinant DNA procedures in which the antibody fragment is linked either directly or via a coupling agent to the effector molecule. Techniques for conjugating such effector molecules to antibodies are well known in the art (see, Hellstrom et al., Controlled Drug Delivery, 2nd Ed., Robinson et al., eds., 1987, pp. 623-53; Thorpe et al., 1982, Immunol. Rev., 62:119-58 and Dubowchik et al., 1999, Pharmacology and Therapeutics, 83, 67-123). Particular chemical procedures include, for example, those described in WO 93/06231, WO 92/22583, WO 89/00195, WO 89/01476 and WO 03/031581. Alternatively, where the effector molecule is a protein or polypeptide the linkage may be achieved using recombinant DNA procedures, for example as described in WO 86/01533 and EP0392745.

The term effector molecule as used herein includes, for example, antineoplastic agents, drugs, toxins, biologically active proteins, for example enzymes, other antibody or antibody fragments, synthetic or naturally occurring polymers, nucleic acids and fragments thereof e.g. DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

Examples of effector molecules may include cytotoxins or cytotoxic agents including any agent that is detrimental to (e.g. kills) cells. Examples include combrestatins, dolastatins, epothilones, staurosporin, maytansinoids, spongistatins, rhizoxin, halichondrins, roridins, hemiasterlins, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

Effector molecules also include, but are not limited to, antimetabolites (e.g. methotrexate, 6-mercaptopurine, 6-thioguanine, cytarabine, 5-fluorouracil decarbazine), alkylating agents (e.g. mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g. daunorubicin (formerly daunomycin) and doxorubicin), antibiotics (e.g. dactinomycin (formerly actinomycin), bleomycin, mithramycin, anthramycin (AMC), calicheamicins or duocarmycins), and anti-mitotic agents (e.g. vincristine and vinblastine).

Other effector molecules may include chelated radionuclides such as ¹¹¹In and ⁹⁰Y, Lu¹⁷⁷, Bismuth²¹³, Californium²⁵², Iridium¹⁹² and Tungsten¹⁸⁸/Rhenium¹⁸⁸; or drugs such as but not limited to, alkylphosphocholines, topoisomerase I inhibitors, taxoids and suramin.

Other effector molecules include proteins, peptides and enzymes. Enzymes of interest include, but are not limited to, proteolytic enzymes, hydrolases, lyases, isomerases, transferases. Proteins, polypeptides and peptides of interest include, but are not limited to, immunoglobulins, toxins such as abrin, ricin A, pseudomonas exotoxin, or diphtheria toxin, a protein such as insulin, tumour necrosis factor, α-interferon, β-interferon, nerve growth factor, platelet derived growth factor or tissue plasminogen activator, a thrombotic agent or an anti-angiogenic agent, e.g. angiostatin or endostatin, or, a biological response modifier such as a lymphokine, interleukin-1 (IL-1), interleukin-2 (IL-2), granulocyte macrophage colony stimulating factor (GM-CSF), granulocyte colony stimulating factor (G-CSF), nerve growth factor (NGF) or other growth factor and immunoglobulins.

Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include ¹²⁵I, ¹³¹I, ¹¹¹In and ⁹⁹Tc.

In another example the effector molecule may increase the half-life of the antibody in vivo, and/or reduce immunogenicity of the antibody and/or enhance the delivery of an antibody across an epithelial barrier to the immune system. Examples of suitable effector molecules of this type include polymers, albumin, albumin binding proteins or albumin binding compounds such as those described in WO05/117984.

In one embodiment a half-life provided by an effector molecule which is independent of CSF-1R is advantageous.

Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero-polysaccharide.

Specific optional substituents which may be present on the above-mentioned synthetic polymers include one or more hydroxy, methyl or methoxy groups.

Specific examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof.

Specific naturally occurring polymers include lactose, amylose, dextran, glycogen or derivatives thereof.

In one embodiment the polymer is albumin or a fragment thereof, such as human serum albumin or a fragment thereof.

“Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

The size of the polymer may be varied as desired, but will generally be in an average molecular weight range from 500 Da to 50000 Da, for example from 5000 to 40000 Da such as from 20000 to 40000 Da. The polymer size may in particular be selected on the basis of the intended use of the product for example ability to localize to certain tissues such as tumors or extend circulating half-life (for review see Chapman, 2002, Advanced Drug Delivery Reviews, 54, 531-545). Thus, for example, where the product is intended to leave the circulation and penetrate tissue, for example for use in the treatment of a tumour, it may be advantageous to use a small molecular weight polymer, for example with a molecular weight of around 5000 Da. For applications where the product remains in the circulation, it may be advantageous to use a higher molecular weight polymer, for example having a molecular weight in the range from 20000 Da to 40000 Da.

Suitable polymers include a polyalkylene polymer, such as a poly(ethyleneglycol) or, especially, a methoxypoly(ethyleneglycol) or a derivative thereof, and especially with a molecular weight in the range from about 15000 Da to about 40000 Da.

In one example antibodies for use in the present invention are attached to poly(ethyleneglycol) (PEG) moieties. In one particular example the antibody is an antibody fragment and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (see for example U.S. Pat. No. 5,219,996; U.S. Pat. No. 5,667,425; WO98/25971, WO2008/038024). In one example the antibody molecule of the present invention is a modified Fab fragment wherein the modification is the addition to the C-terminal end of its heavy chain one or more amino acids to allow the attachment of an effector molecule. Suitably, the additional amino acids form a modified hinge region containing one or more cysteine residues to which the effector molecule may be attached. Multiple sites can be used to attach two or more PEG molecules.

Suitably PEG molecules are covalently linked through a thiol group of at least one cysteine residue located in the antibody fragment. Each polymer molecule attached to the modified antibody fragment may be covalently linked to the sulphur atom of a cysteine residue located in the fragment. The covalent linkage will generally be a disulphide bond or, in particular, a sulphur-carbon bond. Where a thiol group is used as the point of attachment appropriately activated effector molecules, for example thiol selective derivatives such as maleimides and cysteine derivatives may be used. An activated polymer may be used as the starting material in the preparation of polymer-modified antibody fragments as described above. The activated polymer may be any polymer containing a thiol reactive group such as an α-halocarboxylic acid or ester, e.g. iodoacetamide, an imide, e.g. maleimide, a vinyl sulphone or a disulphide. Such starting materials may be obtained commercially (for example from Nektar, formerly Shearwater Polymers Inc., Huntsville, Ala., USA) or may be prepared from commercially available starting materials using conventional chemical procedures. Particular PEG molecules include 20K methoxy-PEG-amine (obtainable from Nektar, formerly Shearwater; Rapp Polymere; and SunBio) and M-PEG-SPA (obtainable from Nektar, formerly Shearwater).

In one embodiment, the antibody is a modified Fab fragment, Fab′ fragment or diFab which is PEGylated, i.e. has PEG (poly(ethyleneglycol)) covalently attached thereto, e.g. according to the method disclosed in EP 0948544 or EP1090037 [see also “Poly(ethyleneglycol) Chemistry, Biotechnical and Biomedical Applications”, 1992, J. Milton Harris (ed), Plenum Press, New York, “Poly(ethyleneglycol) Chemistry and Biological Applications”, 1997, J. Milton Harris and S. Zalipsky (eds), American Chemical Society, Washington D.C. and “Bioconjugation Protein Coupling Techniques for the Biomedical Sciences”, 1998, M. Aslam and A. Dent, Grove Publishers, New York; Chapman, A. 2002, Advanced Drug Delivery Reviews 2002, 54:531-545]. In one example PEG is attached to a cysteine in the hinge region. In one example, a PEG modified Fab fragment has a maleimide group covalently linked to a single thiol group in a modified hinge region. A lysine residue may be covalently linked to the maleimide group and to each of the amine groups on the lysine residue may be attached a methoxypoly(ethyleneglycol) polymer having a molecular weight of approximately 20,000 Da. The total molecular weight of the PEG attached to the Fab fragment may therefore be approximately 40,000 Da.

Particular PEG molecules include 2-[3-(N-maleimido)propionamido]ethyl amide of N,N′-bis(methoxypoly(ethylene glycol) MW 20,000) modified lysine, also known as PEG2MAL40K (obtainable from Nektar, formerly Shearwater).

Alternative sources of PEG linkers include NOF who supply GL2-400MA3 (wherein m in the structure below is 5) and GL2-400MA (where m is 2) and n is approximately 450:

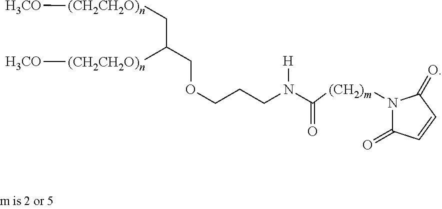

That is to say each PEG is about 20,000 Da.

Thus in one embodiment the PEG is 2,3-Bis(methylpolyoxyethylene-oxy)-1-{[3-(6-maleimido-1-oxohexyl)amino]propyloxy} hexane (the 2 arm branched PEG, —CH₂)₃NHCO(CH₂)₅-MAL, Mw 40,000 known as SUNBRIGHT GL2-400MA3.

Further alternative PEG effector molecules of the following type:

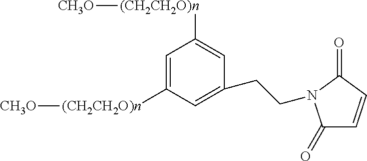

In one embodiment there is provided an antibody, such as a full length antibody, which is PEGylated (for example with a PEG described herein), attached through a cysteine amino acid residue at or about amino acid 226 in the chain, for example amino acid 226 of the heavy chain (by sequential numbering).

In one embodiment the present disclosure provides a Fab′PEG molecule comprising one or more PEG polymers, for example 1 or 2 polymers such as a 40 kDa polymer or polymers.

Fab-PEG molecules according to the present disclosure may be particularly advantageous in that they have a half-life independent of the Fc fragment.

In one embodiment there is provided a scFv conjugated to a polymer, such as a PEG molecule, a starch molecule or an albumin molecule.

In one embodiment the antibody or fragment is conjugated to a starch molecule, for example to increase the half-life. Methods of conjugating start to a protein as described in U.S. Pat. No. 8,017,739 incorporated herein by reference.

To identify inhibitors of CSF-1R activity a number of different approaches may be taken by those skilled in the art. In one example inhibitors are identified by first identifying agents that interact with CSF-1, IL-34 or CSF-1R and subsequently testing those agents to identify those that inhibit CSF-1R activity. In one such example the agent is an antibody.

Agents that interact with CSF-1, IL-34 or CSF1-R may be identified using any suitable method, for example by using a cell-free or cell-based assay system where the CSF-1, IL-34 or CSF-1R polypeptide is contacted with a candidate agent and the ability of the candidate agent to interact with the polypeptide is determined. Preferably, the ability of a candidate agent to interact with a CSF-1, IL-34 or CSF-1R polypeptide is compared to a reference range or control. If desired, this assay may be used to screen a plurality (e.g. a library) of candidate agents using a plurality of CSF-1, IL-34 or CSF-1R polypeptide samples. In one example of a cell free assay, a first and second sample comprising native or recombinant CSF-1, IL-34 or CSF-1R polypeptide are contacted with a candidate agent or a control agent and the ability of the candidate agent to interact with the polypeptide is determined by comparing the difference in interaction between the candidate agent and control agent. Preferably, the polypeptide is first immobilized, by, for example, contacting the polypeptide with an immobilized antibody which specifically recognizes and binds it, or by contacting a purified preparation of polypeptide with a surface designed to bind proteins. The polypeptide may be partially or completely purified (e.g. partially or completely free of other polypeptides) or part of a cell lysate. Further, the polypeptide may be a fusion protein comprising the CSF-1, IL-34 or CSF1-R polypeptide or a biologically active portion thereof and a domain such as glutathionine-S-transferase or the Fc region of IgGl. Alternatively, the polypeptide can be biotinylated using techniques well known to those of skill in the art (e.g. biotinylation kit, Pierce Chemicals; Rockford, Ill.). The ability of the candidate agent to interact with the polypeptide can be determined by methods known to those of skill in the art for example, ELIS A, BIAcore™, Flow cytometry or fluorescent microvolume assay technology (FMAT). In another example where a cell-based assay is used, a population of cells expressing CSF-1, IL-34 or CSF-1 R is contacted with a candidate agent and the ability of the candidate agent to interact with the polypeptide is determined. Preferably, the ability of a candidate agent to interact with CSF-1, IL-34 or CSF-1 R is compared to a reference range or control. The cell, for example, can be of eukaryotic origin (e.g. yeast or mammalian) and can express the CSF-1, IL-34 or CSF-1 R polypeptide endogenously or be genetically engineered to express the polypeptide. In some instances, the CSF-1, IL-34 or CSF-1R polypeptide or the candidate agent is labelled, for example with a radioactive label (such as P, S or I) or a fluorescent label (such as fluorescein isothiocyanate, rhodamine, phycoerythrin, phycocyanin, allophycocyanin, o-phthaldehyde or fluorescamine) to enable detection of an interaction between a polypeptide and a candidate agent. Alternative methods such as ELISA, flow cytometry and FMAT may also be used. Agents which inhibit CSF-1R activity may be identified by any suitable method, for example by: (i) comparing the activity of CSF-1R in the presence of a candidate agent with the activity of said polypeptide in the absence of the candidate agent or in the presence of a control agent; and (ii) determining whether the candidate agent inhibits activity of CSF-1R. Such assays can be used to screen candidate agents, in clinical monitoring or in drug development. As described above, agents may be pre-screened where appropriate (e.g. an antibody) to identify agents that interact with CSF-1, IL-34 or CSF-1R prior to screening those agents which bind for their ability to inhibit CSF-1R activity. In one example a cell-based assay system is used to identify agents capable of inhibiting the activity of CSF-1R.

In one particular example the assay used to identify inhibitors of CSF-1 activity or CSF-1R activity is the standard in vitro colony stimulating assay of Metcalf, 1970, J. Cell. Physiol. 76-89 in which CSF-1 is capable of stimulating the formation of macrophage colonies. Potential inhibitors are added to the assay and proliferation of macrophages is measured by any suitable method such as ³H thymidine incorporation or formazan dye conversion. Inhibition is therefore measured as a reduction in proliferation compared to controls.

In another example inhibitors of CSF-1R may down-regulate the expression of the CSF-1, IL-34 or CSF-1R polypeptide, for example antisense inhibitors. Such inhibitors may be identified by any method known in the art. In one example such inhibitors are identified in a cell-based assay system. Accordingly, a population of cells expressing a CSF-1, IL-34 or CSF-1R polypeptide or nucleic acid are contacted with a candidate agent and the ability of the candidate agent to alter expression of the CSF-1, IL-34 or CSF-1R polypeptide or nucleic acid is determined by comparison to a reference range or control. In one example, populations of cells expressing a CSF-1, IL-34 or CSF1-R polypeptide are contacted with a candidate agent or a control agent and the ability of the candidate agent to alter the expression of the CSF-1, IL-34 or CSF-1R polypeptides or nucleic acids is determined by comparing the difference in the level of expression of the CSF-1, IL-34 or CSF1-R polypeptides or nucleic acids between the treated and control populations of cells. If desired, this assay may be used to screen a plurality (e.g. a library) of candidate agents. The cell, for example, can be of eukaryotic origin (e.g. yeast or mammalian) and can express a CSF-1, IL-34 or CSF-1R polypeptide endogenously or be genetically engineered to express a CSF-1, IL-34 or CSF-1R polypeptide. The ability of the candidate agents to alter the expression of a said polypeptides or nucleic acids can be determined by methods known to those of skill in the art, for example and without limitation, by flow cytometry, radiolabelling, a scintillation assay, immunoprecipitation, Western blot analysis, Northern blot analysis or RT-PCR.

Agents that inhibit the activity of CSF-1R may be identified or further tested, for example to determine therapeutically effective amounts in one or more animal models. Examples of suitable animals include, but are not limited to, mice, rats, rabbits, monkeys, guinea pigs, dogs and cats. In one example where the agent inhibits the expression of CSF-1, IL-34 or CSF-1R, a first and second group of mammals are administered with a candidate agent or a control agent and the ability of the candidate agent to inhibit the expression of CSF-1, IL-34 or CSF-1R polypeptide or nucleic acid is determined by comparing the difference in the level of expression between the first and second group of mammals. Where desired, the expression levels of the CSF-1, IL-34 or CSF-1R polypeptides or nucleic acid in the first and second groups of mammals can be compared to the level of CSF-1, IL-34 or CSF-1R polypeptide or nucleic acid in a control group of mammals. The candidate agent or a control agent can be administered by means known in the art (e.g. orally, rectally or parenterally such as intraperitoneally or intravenously). Changes in the expression of a polypeptide or nucleic acid can be assessed by the methods outlined above. In another example, the inhibition of CSF-1R activity can be determined by monitoring an amelioration or improvement in disease symptoms, and/or a delayed onset or slowed progression of the disease. For example but without limitation, in the case of IPF this could be manifest as a reduction in the decline of lung function as measured by forced vital capacity, a reduction in the decline of diffusing capacity as measured by DLCO, or a reduction in the decline of the six minute walking distance Inhibition of CSF-1R activity in patients with IPF can also be determined by monitoring clinical events such as exacerbations, defined as a rapid deterioration in disease activity in the absence of other causes. In the case of hepatic fibrosis, the enhanced liver fibrosis (ELF) test can be used to monitor disease by quantitatively measuring the amount of hyaluronic acid, amino-terminal pro-peptide of type III pro-collagen (PIIINP) and tissue inhibitor of metalloproteinase 1 (TIMP1) in serum and combining them through use of an algorithm to produce a single ELF score, the magnitude of which indicates disease severity. With regard to systemic sclerosis, the effects of CSF-1R inhibition could be manifest as reduced progression of the Modified Rodnan Skin Score and/or the Medsger Severity Scale, or at the level of individual organs, for example retardation of scleroderma associated interstitial lung disease. The effect of CSF-1R inhibition on the course of renal fibrosis, including but not limited to chronic kidney disease and post-transplant graft fibrosis, could be manifest as a reduction in the decline or improvement of glomerular filtration rate (GFR) and/or proteinuria.

Thus in one embodiment employing an inhibitor of CSF-1R may provide a reduction in one or more of the follow in the decline of lung function as measured by forced vital capacity or a reduction in the decline of distance covered in the six minute walk test in IPF patients. The enhanced liver fibrosis (ELF) test can be used to monitor liver fibrosis by quantitatively measuring the amount of hyaluronic acid, amino-terminal pro-peptide of type III pro-collagen (PIIINP) and tissue inhibitor of metalloproteinase 1 (TIMP1) in serum and combining them through use of an algorithm to produce a single ELF score, the magnitude of which indicates disease severity.

Techniques known to physicians familiar with fibrotic disease can be used to determine whether a candidate agent has altered one or more symptoms associated with the disease. A number of different models of fibrotic disease are known in the art. These include bleomycin-induced lung fibrosis, reviewed in Moore B B and Hogaboam C M, 2008, Am J Physiol Lung Cell Mol Physiol, 294, L152-60; Scotton C J and Chambers R C, 2010 Am J Physiol Lung Cell Mol Physiol, 299, L439-41, adenosine deaminase deficient mouse model of adenosine-induced fibrosis (Chunn J L et al, 2005, J Immunol 175: 1937-46; Sun C_X et al, 2006, J Clin Invest, 116, 2173-82), radiation induced lung fibrosis, (Sharplin J and Franko A J, 1989, Radiation Research 119, 1-14; Sharplin J and Franko A J, 1989, Radiation Research 119, 15-31; Johnston C J et al, 1998, Exp Lung Res, 24, 321-37; Haston C K and Travis E L, 1997, Cancer Res 57, 5286-91; Katoh H et al, 2010, J Radiat Res, 51, 325-332), FITC induced lung fibrosis (Roberts S N et al, 1995, J Pathol, 176, 309-18; Christensen, P et al, 1999, Am J Pathol, 155, 1773-79), silica induced fibrosis in the mouse (Davis G S, et al, 1998, J Environ Pathol Toxicol Oncol 17, 81-97; Lakatos H F et al, 2006, Exp Lung Res 32, 181-99), the carbon tetrachloride (CCl4) model of liver fibrosis (Iredale J P et al, 1998, J Clin Invest, 102, 538-49), the bile duct ligation model of hepatic fibrosis (Stahelin B J et al, 1999, Virchows Arch, 434, 333-39; Issa R et al, 2001, Gut, 48, 548-57), the UUO (unilateral ureteric obstruction) of kidney fibrosis (Klahr S and Pukerson M L, 1994, Am J Kidney Dis, 23, 219-23; Satoh M et al, 2001, J Am Soc Nephrol, 12, 317-25), the adriamycin induced model of chronic kidney disease (Tamaki, K et al, 1994, Kidney Int, 45, 525-36; Wang Y et al, 2000, Kidney Int, 58, 1797-1804 and reviewed by Lee V W and Harris D C, 2011, Nephrology, 16, 30-38), the 5/6 reduction model of chronic kidney disease (Griffin A et al, 1994, J Am Soc Nephrol, 4, 2023-31; the hypochlorous acid induced model of systemic sclerosis (Servettaz, A et al, 2009, J Immunol, 182(9), 5855-64), and alternative models of systemic sclerosis such as bleomycin induced skin fibrosis, tight skin mouse model, in which mice are heterozygous for a mutation in the fibrillin-1 gene and the sclerodermatous graft versus host disease model where the mice develop skin and pulmonary fibrosis, reviewed in Yamamoto, T, 2010, J Dermatol, 37, 26-41. Generally the different animal models exhibit excessive production and deposition of extracellular matrix including collagen in the affected organ or tissue which is the hallmark of fibrosis. Other measurements of fibrotic disease will vary between models as different organs are affected in the specific models. The elevation of protein and creatinine levels in urea may be measured in models of kidney fibrosis whilst in models of pulmonary fibrosis a histopathological score of the extent and severity of fibrosis, for example, the modified Ashcroft score, is often used (Hubner R H et al, 2008, Biotechniques 44: 507-17).

Monitoring techniques for fibrosis include lung function tests for pulmonary fibrosis, the ELF test for liver disease/fibrosis, the FibroTest (blood analysis of fibrosis markers) for liver disease and transient elastography (Fibroscan®) which is an imaging technology that provides a measure of liver stiffness, useful in detecting and assessing liver fibrosis. Analysis of tissue samples from a biopsy can also be performed.

As discussed herein, inhibitors of CSF-1R activity can be used in the treatment and/or prophylaxis of fibrotic disease. For such use the agents will generally be administered in the form of a pharmaceutical composition. Also provided is a pharmaceutical composition comprising an inhibitor of CSF-1R activity and a pharmaceutically acceptable diluent, excipient and/or carrier. The term ‘treatment’ includes either therapeutic or prophylactic therapy. When a reference is made herein to a method of treating or preventing a disease or condition using a particular inhibitor or combination of inhibitors, it is to be understood that such a reference is intended to include the use of that inhibitor or combination of inhibitors for the manufacture of a medicament for the treatment and/or prophylaxis of fibrotic disease. The composition will usually be supplied as part of a sterile, pharmaceutical composition that will normally include a pharmaceutically acceptable carrier. This composition may be in any suitable form (depending upon the desired method of administering it to a patient). The inhibitors of use in the invention are preferably administered to a subject orally or intrarectally but may also be administered by a variety of other routes such as transdermally, subcutaneously, intranasally, intravenously and intramuscularly. The most suitable route for administration in any given case will depend on the particular inhibitor, the subject, and the nature and severity of the disease and the physical condition of the subject.

The inhibitors of use in the invention may be administered in combination, e.g. simultaneously, sequentially or separately, with one or more other therapeutically active compounds, which may be for example an anti-fibrotic therapy such as Perfenidone or anticancer therapies.

Pharmaceutical compositions maybe conveniently presented in unit dose forms containing a predetermined amount of an active agent of the invention per dose. Such a unit may contain for example but without limitation, 1000 mg/kg to 0.01 mg/kg for example 750 mg/kg to 0.1 mg/kg, such as 100 mg/kg to 1 mg/kg depending on the condition being treated, the route of administration and the age, weight and condition of the subject.

Pharmaceutically acceptable carriers for use in the invention may take a wide variety of forms depending, e.g. on the route of administration.

Compositions for oral administration may be liquid or solid. Oral liquid preparations may be in the form of, for example, aqueous or oily suspensions, solutions, emulsions, syrups or elixirs, or may be presented as a dry product for reconstitution with water or other suitable vehicle before use. Oral liquid preparations may contain suspending agents as known in the art. In the case of oral solid preparations such as powders, capsules and tablets, carriers such as starches, sugars, microcrystalline cellulose, granulating agents, lubricants, binders, disintegrating agents, and the like may be included. Because of their ease of administration, tablets and capsules represent the most advantageous oral dosage unit form in which case solid pharmaceutical carriers are generally employed.

In addition to the common dosage forms set out above, active agents of the invention may also be administered by controlled release means and/or delivery devices. Tablets and capsules may comprise conventional carriers or excipients such as binding agents for example, syrup, acacia, gelatin, sorbitol, tragacanth, or polyvinylpyrrolidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulphate. The tablets may be coated by standard aqueous or non-aqueous techniques according to methods well known in normal pharmaceutical practice. Pharmaceutical compositions of the present invention suitable for oral administration may be presented as discrete units such as capsules, cachets or tablets, each containing a predetermined amount of the active agent, as a powder or granules, or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active agent with the carrier, which constitutes one or more necessary ingredients. In general, the compositions are prepared by uniformly and intimately admixing the active agent with liquid carriers or finely divided solid carriers or both, and then, if necessary, shaping the product into the desired presentation. For example, a tablet may be prepared by compression or moulding, optionally with one or more accessory ingredients.

Pharmaceutical compositions suitable for parenteral administration may be prepared as solutions or suspensions of the active agents of the invention in water suitably mixed with a surfactant such as hydroxypropylcellulose. Dispersions can also be prepared in glycerol, liquid polyethylene glycols, and mixtures thereof in oils. Under ordinary conditions of storage and use, these preparations contain a preservative to prevent the growth of microorganisms. The pharmaceutical forms suitable for injectable use include aqueous or non-aqueous sterile injection solutions which may contain anti-oxidants, buffers, bacteriostats and solutes which render the composition isotonic with the blood of the intended recipient, and aqueous and non-aqueous sterile suspensions which may include suspending agents and tWckening agents. Extemporaneous injection solutions, dispersions and suspensions may be prepared from sterile powders, granules and tablets. Pharmaceutical compositions can be administered with medical devices known in the art. For example, in a preferred embodiment, a pharmaceutical composition of the invention can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

Pharmaceutical compositions adapted for topical administration may be formulated as ointments, creams, suspensions, lotions, powders, solutions, pastes, gels, impregnated dressings, sprays, aerosols or oils, transdermal devices, dusting powders, and the like. These compositions may be prepared via conventional methods containing the active agent. Thus, they may also comprise compatible conventional carriers and additives, such as preservatives, solvents to assist drug penetration, emollients in creams or ointments and ethanol or oleyl alcohol for lotions. Such carriers may be present as from about 1 percent up to about 98 percent of the composition. More usually they will form up to about 80 percent of the composition. As an illustration only, a cream or ointment is prepared by mixing sufficient quantities of hydrophilic material and water, containing from about 5-10 percent by weight of the compound, in sufficient quantities to produce a cream or ointment having the desired consistency. Pharmaceutical compositions adapted for transdermal administration may be presented as discrete patches intended to remain in intimate contact with the epidermis of the recipient for a prolonged period of time. For example, the active agent may be delivered from the patch by iontophoresis. For applications to external tissues, for example the mouth and skin, the compositions are preferably applied as a topical ointment or cream. When formulated in an ointment, the active agent may be employed with either a paraffinic or a water-miscible ointment base. Alternatively, the active agent may be formulated in a cream with an oil-in-water cream base or a water-in-oil base. Pharmaceutical compositions adapted for topical administration in the mouth include lozenges, pastilles and mouth washes. Pharmaceutical compositions adapted for topical administration to the eye include eye drops wherein the active agent is dissolved or suspended in a suitable carrier, especially an aqueous solvent. They also include topical ointments or creams as above. Pharmaceutical compositions suitable for rectal administration wherein the carrier is a solid are most preferably presented as unit dose suppositories. Suitable carriers include cocoa butter or other glyceride or materials commonly used in the art, and the suppositories may be conveniently formed by admixture of the combination with the softened or melted capier(s) followed by chilling and shaping moulds. They may also be administered as enemas.

The dosage to be administered of an inhibitor of CSF-1R activity will vary according to the particular inhibitor, the type of fibrotic disease, the subject, and the nature and severity of the disease and the physical condition of the subject, and the selected route of administration; the appropriate dosage can be readily determined by a person skilled in the art. For the treatment and/or prophylaxis of fibrotic disease in humans and animals pharmaceutical compositions comprising antibodies can be administered to patients (e.g., human subjects) at therapeutically or prophylactically effective dosages (e.g. dosages which result in inhibition of fibrotic disease and/or relief of fibrotic disease symptoms) using any suitable route of administration, such as injection and other routes of administration known in the art for clinical products, such as antibody-based clinical products. The compositions may contain at least 0.05 percent by weight, for example 0.5 to 50 percent by weight such as 1 to 10 percent by weight, or more, by weight, of the inhibitor of the invention, depending on the method of administration. It will be recognized by one of skill in the art that the optimal quantity and spacing of individual dosages of an inhibitor of the invention will be determined by the nature and extent of the condition being treated, the form, route and site of administration, and the age and condition of the particular subject being treated, and that a physician will ultimately determine appropriate dosages to be used. This dosage may be repeated as often as appropriate. If side effects develop the amount and/or frequency of the dosage can be altered or reduced, in accordance with normal clinical practice.

In another example, where the inhibitor is a nucleic acid this may be administered via gene therapy (see for example Hoshida, T. et al, 2002, Pancreas, 25:111-121; Ikuno, Y. 2002, Invest. Ophthalmol. Vis. Sci. 2002 43:2406-2411; Bollard, C, 2002, Blood 99:3179-3187; Lee E., 2001, Mol. Med. 7:773-782). Gene therapy refers to administration to a subject of an expressed or expressible nucleic acid, in one example this is either the CSF-1, IL-34 or the CSF-IR nucleic acid or portions thereof. Any of the methods for gene therapy available in the art can be used according to the present invention. Delivery of the therapeutic nucleic acid into a patient can be direct in vivo gene therapy (i.e. the patient is directly exposed to the nucleic acid or nucleic acid-containing vector) or indirect ex vivo gene therapy (i.e. cells are first transformed with the nucleic acid in vitro and then transplanted into the patient). For example for in vivo gene therapy, an expression vector containing the CSF-1, IL-34 or CSF-IR nucleic acid may be administered in such a manner that it becomes intracellular, i.e. by infection using a defective or attenuated retroviral or other viral vectors as described, for example, in U.S. Pat. No. 4,980,286 or by Robbins et al, 1998, Pharmacol. Ther. 80:35-47. The various retroviral vectors that are known in the art are such as those described in Miller et al. (1993, Meth. Enzymol. 217:581-599) which have been modified to delete those retroviral sequences which are not required for packaging of the viral genome and subsequent integration into host cell DNA. Also adenoviral vectors can be used which are advantageous due to their ability to infect non-dividing cells and such high-capacity adenoviral vectors are described in Kochanek (1999, Human Gene Therapy, 10:2451-2459). Chimeric viral vectors that can be used are those described by Reynolds et al. (1999, Molecular Medicine Today, 1:25-31). Hybrid vectors can also be used and are described by Jacoby et al. (1997, Gene Therapy, 4:1282-1283). Direct injection of naked DNA or through the use of microparticle bombardment (e.g. Gene Gun(R); Biolistic, Dupont) or by coating it with lipids can also be used in gene therapy. Cell-surface receptors/transfecting compounds or through encapsulation in liposomes, microparticles or microcapsules or by administering the nucleic acid in linkage to a peptide which is known to enter the nucleus or by administering it in linkage to a ligand predisposed to receptor-mediated endocytosis (See Wu and Wu, 1987, J. Biol. Chem., 262:4429-4432) can be used to target cell types which specifically express the receptors of interest. In ex vivo gene therapy, a gene is transferred into cells in vitro using tissue culture and the cells are delivered to the patient by various methods such as injecting subcutaneously, application of the cells into a skin graft and the intravenous injection of recombinant blood cells such as haematopoietic stem or progenitor cells. Cells into which a CSF-1, IL-34 or CSF-IR nucleic acid can be introduced for the purposes of gene therapy include, for example, epithelial cells, endothelial cells, keratinocytes, fibroblasts, muscle cells, hepatocytes and blood cells. The blood cells that can be used include, for example, T-lymphocytes, B-lymphocytes, monocytes, macrophages, neutrophils, eosinophils, megakaryotcytes, granulocytes, haematopoietic cells or progenitor cells, and the like.

In one aspect, the pharmaceutical composition of the present invention comprises a CSF-1, IL-34 or CSF-IR nucleic acid, said nucleic acid being part of an expression vector that expresses a CSF1, IL-34 or CSF-IR polypeptide or chimeric protein thereof in a suitable host. In particular, such a nucleic acid has a promoter operably linked to the polypeptide coding region, said promoter being inducible or constitutive (and, optionally, tissue-specific).

The invention will now be described with reference to the following examples, which are merely illustrative and should not in any way be construed as limiting the scope of the present invention.

### FIGURES

FIG. 1A shows the amino acid sequence for human CSF-1R

FIG. 1B shows the amino acid sequence for certain variants of CSF-1R

FIG. 1C shows an amino acid sequence of CSF-1R suitable for expression on cell surfaces

FIG. 2 shows flow cytometry data confirming that receptor-bound CSF-1 on M-NFS-60 cells was detectable by this method

FIG. 3 shows Ab535, either immobilised on the surface of the tissue culture well, or free in solution, did not support the proliferation of M-NFS-60 cells in the absence of CSF-1 at the concentrations used

FIG. 4a shows that treatment of bleomycin-induced lung fibrosis with Ab535 reduced the amount of BAL fluid collagen compared to treatment with the isotype control.

FIG. 4b shows that treatment of bleomycin-induced lung fibrosis with Ab535 reduced the Ashcroft score of the samples compared to treatment with the isotype control demonstrating that the mice treated with Ab535 had improved fibrotic pathology.

FIG. 4c shows that treatment of bleomycin-induced lung fibrosis with Ab535 reduced the amount of albumin in BAL fluid compared to treatment with the isotype control which thus shows that the integrity of the pulmonary barrier of the mice treated with Ab535 was improved.

FIG. 4d shows that treatment of bleomycin-induced lung fibrosis with Ab535 reduced the number of macrophages in the BAL fluid.

FIG. 5 shows representative images of the histopathological analysis of lungs from saline control, bleomycin plus isotype control and bleomycin plus Ab 535 treated animals. Animals treated with Ab 535 had a greatly reduced fibrosis of the lungs compared to the isotype control, bleomycin treated animals.

FIG. 6a shows that treatment of ADA-deficient mice with induced lung fibrosis, with Ab535, reduced BALF collagen concentration compared to treatment with the isotype control.

FIG. 6b shows that treatment of ADA-deficient mice with induced lung fibrosis, with Ab535, reduced the Ashcroft score of the samples compared to treatment with the isotype control demonstrating that the mice treated with Ab535 had improved fibrotic pathology.

FIG. 6c shows that treatment of ADA-deficient mice with induced lung fibrosis, with Ab535, reduced the amount of albumin in the BAL compared to treatment with the isotype control demonstrating that the integrity of the pulmonary barrier of the mice treated with Ab535 was improved.

FIG. 6d shows that treatment of ADA-deficient mice with induced lung fibrosis, with Ab535, reduced the number of macrophages in the BAL fluid.

FIG. 7 shows representative images of the histopathological analysis of lung tissue from ADA knockout mice and wild type mice either treated with Ab535 or an isotype control antibody (mouse IgG1). Lung tissue sections were stained with Masson's trichrome. Treatment of ADA knockout mice with Ab535 greatly reduced the amount of fibrotic tissue and collagen deposition.

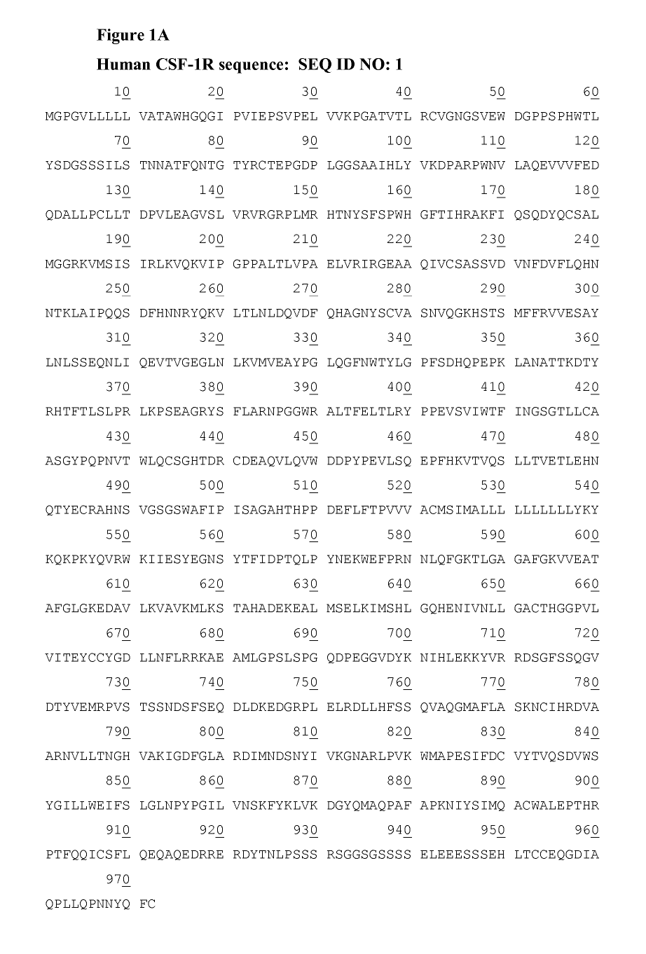

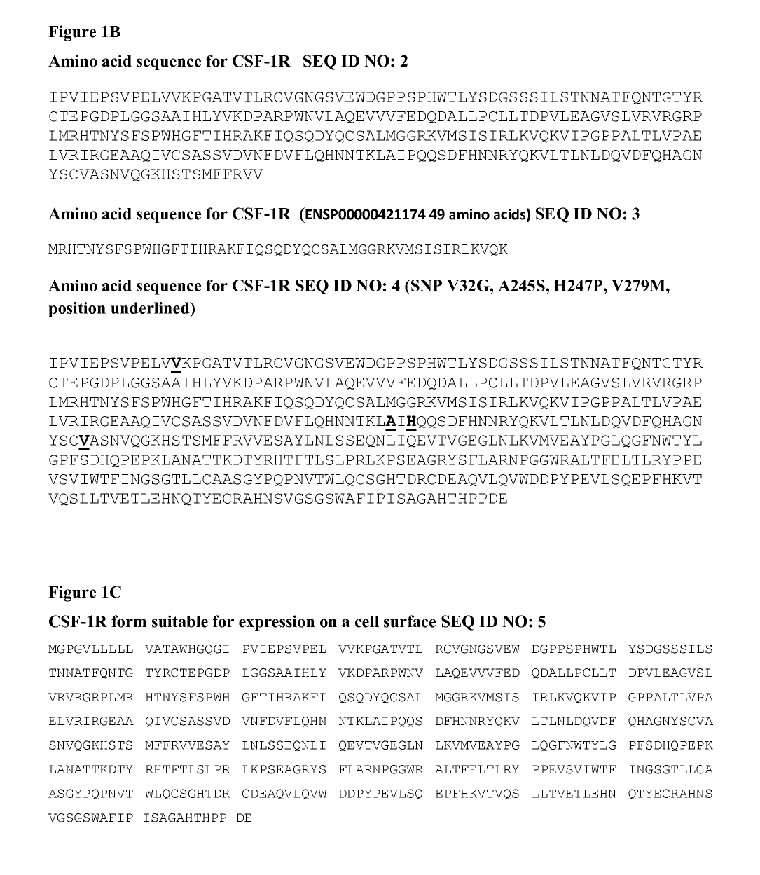

### EXAMPLE 1

### Isolation of an Anti-Mouse CSF-1R Antibody

2 rabbits received 5 immunisations with cells transiently expressing residues 1-512 of mouse CSF-1R (Uniprot Entry p 09581). Antibody response was monitored in an ELISA using Nunc Maxisorp plates coated with 2 μg/ml mouse CSF-1R-Rabbit Fc. Sera titres out to 1:100,000 dilution were observed with both rabbits. Binding of sera to M-NFS-60 cells (Metcalf et al., 1970) was also determined by FACS. Median FL1 was plotted against antibody dilution. Binding out to a dilution of 1:10,000 was observed.

Using the CSF-1-dependent M-NFS-60 cell line (Metcalf et al., 1970), it was shown that sera from both rabbits could block CSF-1-dependent cell survival out to a dilution of 1:100 (data not shown).

One hundred 96-well plates were seeded with 1000-5000 rabbit PBMCs per well and grown for 1 week at 37 C in the presence of EL4-B5 mouse thymoma cells and rabbit T cell conditioned media (TSN). Antibody-containing supernatants were then screened in an FMAT assay using M-NFS-60 cells and anti-rabbit Fc-specific Cy5 conjugate. 678 CSF-1R-binders were identified. From these, approximately 3% blocked CSF-1-dependent M-NFS-60 proliferation in a cell assay. Binders were also screened in an ELISA using plates coated with 2 μg/ml CSF-1R-rabbit Fc and binding revealed with an anti-rabbit F(ab′)2-HRP conjugate. Binders were also screened in an ELISA-based solid-phase blocking assay. For this assay, Nunc Maxisorp 384-well plates were coated with 0.5 μg/ml CSF-1R-rabbit Fc and subsequently blocked in PBS/0.1% Tween-20/1% PEG20000. Plates were washed before culture supernatant was then added to the plates and incubated for >1 hour at room temperature. CSF-1 was then added into the supernatant at a final concentration of 10 ng/ml and incubated for a further 1 hour at room temperature. Plates were washed and then incubated with 0.5 μg/ml biotinylated goat anti-mouse CSF-1 antibody (R&D systems). CSF-1 binding to receptor was revealed using streptavidin-HRP. Eight wells were selected for progression. All demonstrated binding to cells and protein and blocked in the M-NFS-60 assay and in the ELISA.

A fluorescence-based method was used to identify the antigen-specific B cells from a positive well Immunoglobulin-secreting B cells from a positive well were mixed with streptavidin beads coated with biotinylated mouse CSF-1R-rabbit Fc and a goat F(ab′)2 anti-rabbit F(ab′)2 fragment-specific FITC conjugate (Jackson ImmunoResearch). After incubation at 37 C for 1 hour, antibody secreted from antigen-specific B cells was captured on beads in the vicinity of that B cell. The presence of the FITC conjugate resulted in the labelling of antibody-coated beads and the formation of fluorescent foci around the antigen-specific B cells. These individual B cells, identified using a fluorescent microscope, were then picked with a micromanipulator and deposited into a PCR tube. Antibody variable region genes were recovered from single cells by reverse transcription (RT)-PCR. Rabbit V-regions were then expressed as human IgG4 chimeric antibodies or Fabs in a CHO transient expression system. 4 out of 8 wells produced recombinant anti-CSF-1R antibodies. Neutralisation activity with the recombinant antibodies was then confirmed in the M-NFS-60 assay. BIAcore was also performed using the Fab molecules to determine affinity for CSF-1R rabbit Fc (Table 1). Based on neutralisation activity and affinity, Ab535 was selected as the anti-mouse CSF-1R reagent. This antibody was subsequently murinised, expressed in a mammalian system as full length murine IgG1 and purified.

### BIAcore Method

Antibodies were tested for their ability to bind CSF-1R in a BIAcore assay by measurement of binding kinetics to a purified recombinant CSF-1R/Fc fusion protein.

The assay format was capture of the anti-CSF-1R antibodies by immobilised anti-human IgG,F(ab′)₂, then a titration of hCSF-1R/Fc over the captured surface. BIA (Biamolecular Interaction Analysis) was performed using a BIAcore 3000 (GE Healthcare Bio-Sciences AB). All experiments were performed at 25° C. Affinipure F(ab′)₂ fragment goat anti-human IgG, F(ab′)₂ fragment specific (Jackson ImmunoResearch) was immobilised on a CM5 Sensor Chip (GE Healthcare Bio-Sciences AB) via amine coupling chemistry to a level of ˜6000 response units (RU). HBS-EP buffer (10 mM HEPES pH 7.4, 0.15 M NaCl, 3 mM EDTA, 0.005% Surfactant P20, GE Healthcare Bio-Sciences AB) was used as the running buffer with a flow rate of 10 μl/min. An injection of an anti-CSF-1R antibody was performed to give a capture level of approximately 100 RU on the immobilised anti-human IgG,F(ab)₂.

Recombinant human CSF-1R/Fc was titrated (R&D Systems), by doubling dilution, from 2.5 nM to 78 pM over the captured anti-CSF-1R antibody at a flow rate of 30 μl/min for 3 min followed by a 8 min dissociation phase. These sensorgrams were used to generate the association rate. The surface was regenerated at a flow rate of 10 μl/min by two sequential 10 μl injections of 40 mM HCl followed by a 5 μl injection of 10 mM NaOH. Double referenced background subtracted binding curves were analysed using the BIAevaluation software (version 4.1) following standard procedures. Kinetic parameters were determined from the fitting algorithm.

The results for Ab553 are shown in Table 1.

TABLE 1

| ANTIBODY | On Rate | Off Rate | Affinity |
|---|---|---|---|
| REF: | ka (M⁻¹s⁻¹) | kd (s⁻¹) | Constant K_{D} |
| Ab 535 | 8.05 ± 0.01e6 | 1.21 ± 0.15e−5 | 1.50 pM |

### EXAMPLE 2

### In Vitro Analysis of Ab535

### Ab535 Blocks Binding of Murine CSF-1 to CSF-1 Receptor-Positive Cell Lines In Vitro

The capacity of receptor-bound Ab535 to prevent CSF-1 from binding to CSF-1R was investigated. A murine CSF-1R-positive cell line, M-NFS-60, was used in assay in which cells were pre-incubated with Ab535 or a control antibody prior to exposure to CSF-1. Receptor-bound CSF-1 was then detected using a fluorescently-labelled antibody and flow cytometry. An Ab535-dependent decrease in cell fluorescence intensity was interpreted as indicating a capacity of receptor-bound Ab535 to prevent the binding of CSF-1 to CSF-1R.

M-NFS-60 cells (LGC Promochem, Teddington, UK) were maintained in suspension in basal RPMI medium (Invitrogen) supplemented with 10% fetal calf serum (PAA), Hepes (Invitrogen, 10 mM final concentration), Sodium Pyruvate (Invitrogen, 1 mM final concentration) Glucose (Sigma-Aldrich, 4.5 g/l final concentration), Sodium bicarbonate (Sigma-Aldrich, 1.5% final concentration), beta-mercaptoethanol (Sigma-Aldrich, 0.05 mM final concentration), recombinant murine CSF-1 (Preprotech, 3.3 ng/ml final concentration).

M-NFS-60 cells were prepared in flow buffer (PBS (Invitrogen) supplemented with 0.2% BSA (Sigma-Aldrich), 0.09% sodium azide (Sigma-Aldrich)) at a concentration of 1×10E6 cells/ml. Serial dilutions of Ab535 or a murine IgG1 isotype control antibody were prepared in flow buffer and added to 100 μl aliquots of cells to achieve final concentrations of 20, 5, 1.25, 0.32, 0.08 μg/ml in 200 μl volume and were incubated on ice for 1 hour. Thereafter, cells were washed twice in flow buffer, incubated with 0.5 μg/ml recombinant murine CSF-1 for 30 mins on ice and washed twice in flow buffer. To detect receptor-bound CSF-1, cells were incubated with a biotinylated anti-murine CSF-1 antibody (R&D Systems, 5 μg/ml) for 30 minutes on ice, washed twice in flow buffer and bound antibody labelled by incubation with Alexafluor 488-conjugated straptavidin (Invitrogen, 1:200 dilution) on ice for 15 minutes prior to a final washing step and resuspension of cells in 500 μl flow buffer. Cells were assessed for fluorescence by flow cytometry using a FACSCaliber flow cytometer (Becton Dickinson) and data were analysed using WinMDI software.

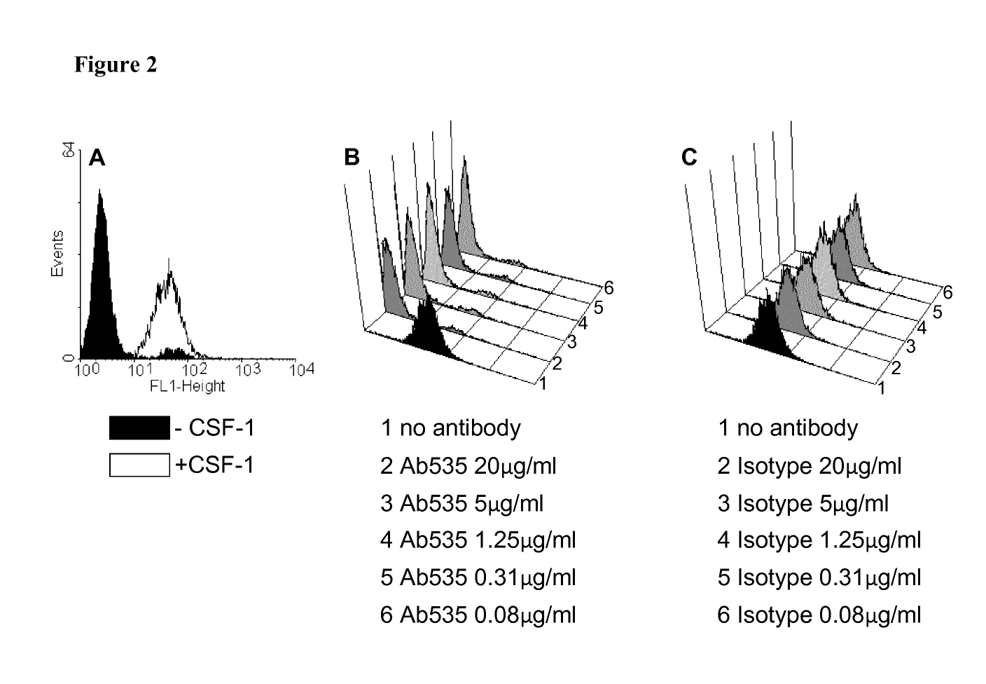

Analysis of flow cytometry data confirmed that receptor-bound CSF-1 on M-NFS-60 cells was detectable by this method (FIG. 2a). Incubation of cells with Ab535 at any of the concentrations used, prior to addition of CSF-1 prevented binding of CSF-1 to CSF-1R as (FIG. 2b). Incubation of cells with an isotype control antibody at any of the concentrations used had no effect on CSF-1 binding and detection (FIG. 2c).

Binding of Ab535 to CSF-1R does not Substitute for CSF-1 in the Proliferation and Survival of a CSF-1 Dependent Cell Line In Vitro

The capacity of Ab535 to substitute for CSF-1 in supporting the survival and proliferation of CSF-1-dependent cells was investigated using M-NFS-60 cells in a cell proliferation assay that included exposing cells to Ab535 free in solution and immobilised on plastic.

To assess the capacity of immobilised Ab535 to support CSF-1-dependent cells, wells of a 96-well round-bottomed tissue culture plate were pre-coated with Ab535 or an irrelevant isotype control antibody for 24 hours prior to initiating the cell proliferation assay by addition of 50 μl per well of a solution containing 10 μg/ml or 1 μg/ml of antibody in PBS and incubation at 4° C. Wells were aspirated and unbound antibody removed by washing each well twice with 100 μl PBS.

Proliferating M-NFS-60 cells, maintained as described above, were seeded into 96-well round-bottomed tissue culture plates at a density of 10,000 cells per well in 50 μl of growth medium without CSF-1. A further 50 μl growth medium, supplemented with either recombinant murine CSF-1, Ab535 or with no supplements, was added to appropriate wells. Plates were incubated for 72 hours and cell number was determined using a CellTiter Glo kit according to the manufacturer's instructions (Promega) to generate a measurable luminescent readout proportional to levels of ATP and hence to cell number, for each of the conditions tested. All conditions were performed in triplicate.

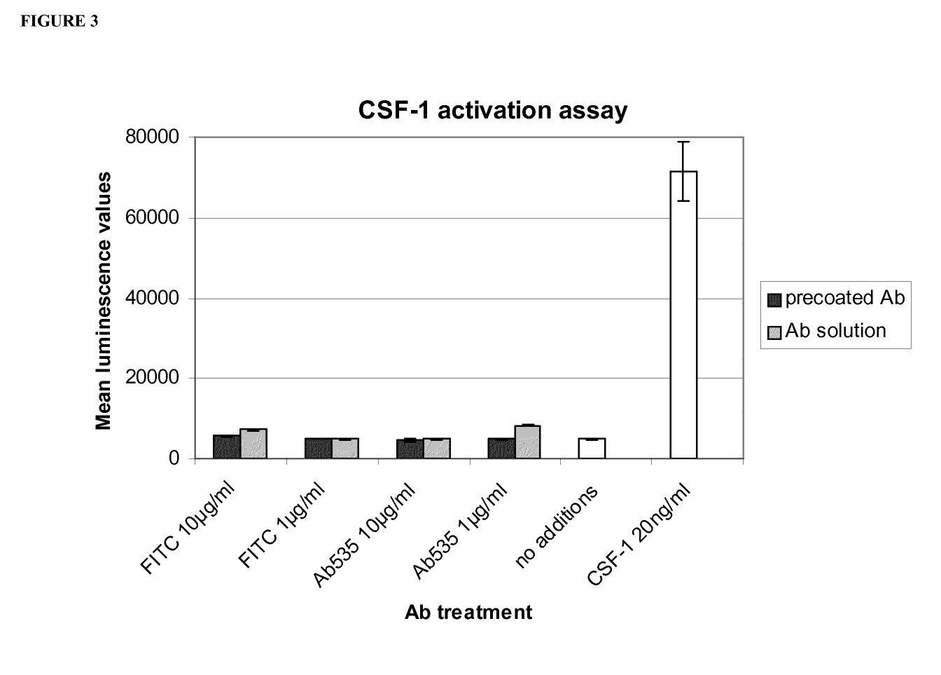

Analysis of the data confirmed that M-NFS-60 are dependent on CSF-1 for proliferation, as reflected by the significantly higher cell number in wells supplemented with CSF-1 compared with those maintained in growth medium without CSF-1. Furthermore, Ab535, either immobilised on the surface of the tissue culture well, or free in solution, did not support the proliferation of M-NFS-60 cells in the absence of CSF-1 at the concentrations used (FIG. 3).

### EXAMPLE 3

### Effect of Anti-CSF-1R Antibody in a Bleomycin Induced In Vivo Model of Pulmonary Fibrosis

Bleomycin is an antibiotic first isolated from Streptomyces verticillatus and has been used as a chemotherapeutic for various cancers. The bleomycin model of lung fibrosis is a well-established model and was used essentially as described in Madtes, D K et al, 1999, Am J Respir Cell Mol Biol, 20, 924-34. The detailed protocol can be found in the following reference Morschl, E., Molina, J. G., Volmer, J., Mohsenin, A., Pero, R. S., Hong, J. S., Kheradmand, F., Lee, J. J. and Blackburn, M. R. (2008), A3 adenosine receptor signaling influences pulmonary inflammation and fibrosis. Am. J. Respir. Cell Mol. Biol. 39: 697-705.

All mice used were wild type C57Blk6 female mice (20 g) purchased from Harlan Labs. An intra-tracheal (IT) cut down instillation was used where mice were anaesthetised with avertin and a tracheostomy performed in order to instil a 3.5 Unit dose of bleomycin in 50 μl of saline or 50 μl of saline alone as a control. Treatment in this manner leads to an inflammatory phase that peaks on day 7 after bleomycin exposure and a fibrotic phase that is maximum on day 21 post exposure. The effect of antibody Ab535 was investigated where antibody was dosed only in the fibrotic phase of the model and was administered subcutaneously at 30 mg/kg, three times per week from day 9-21. Animals were sacrificed at day 21 and readouts included histopathological analysis of the lungs, BAL (bronchoalveolar lavage) fluid cellularity and the measurement of soluble collagen in BAL fluid. Histopathological analysis was conducted to score lung damage using a modified Ashcroft scoring system to determine the severity of lung fibrosis (Hubner R H et al, 2008, Biotechniques 44: 507-17). Excised lungs were inflated with 10% formalin to 25 cm pressure and processed through a series of alcohols and xylene, embedded in paraffin and tissue sections de-paraffinised prior to processing and staining with Masson's Trichrome.

The amount of soluble collagen in BAL fluid was assessed using a commercially available Sircol assay kit following the manufacturer's instructions.
The effect on the macrophage population in the BAL fluid was determined using cytospin preparations. Aliquots of BAL cells were spun onto microscope slides, stained with Diff-Quick and macrophages counted.

It was found that therapeutic treatment with the anti-CSF-1R antibody, Ab535, with dosing started at day 9 resulted in greatly reduced bleomycin-induced lung fibrosis. Both the severity and extent of fibrosis was significantly reduced. The treated mice had reduced collagen production, improved fibrotic pathology and improved pulmonary barrier protection.

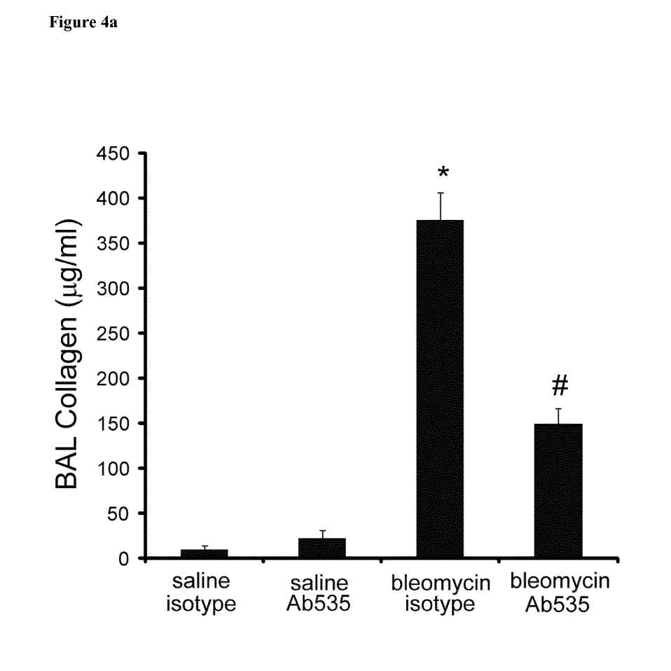

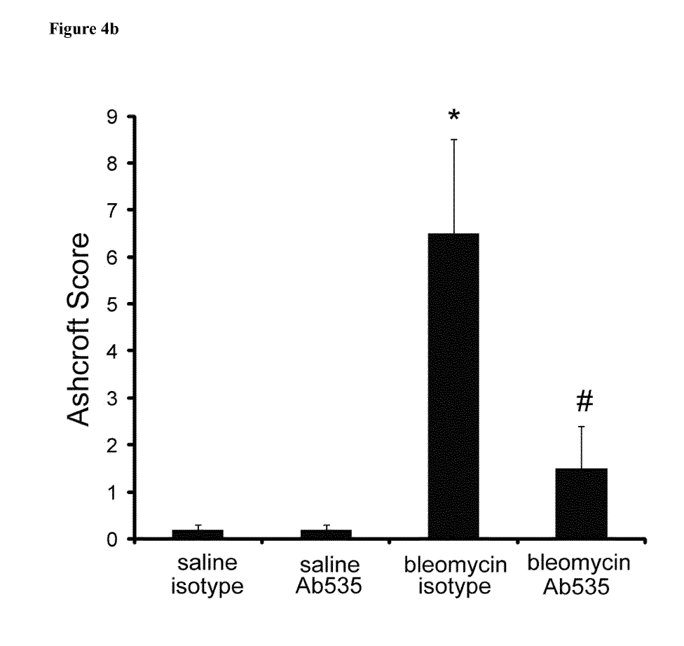

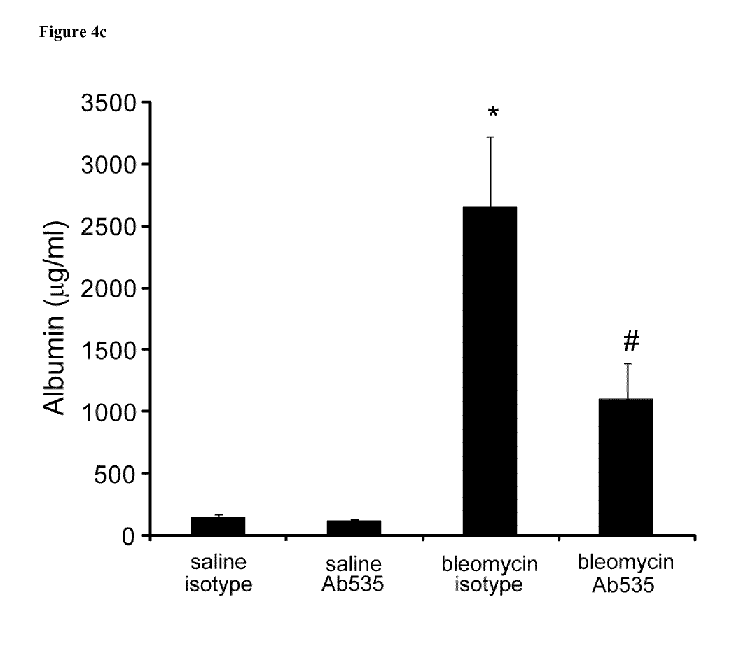

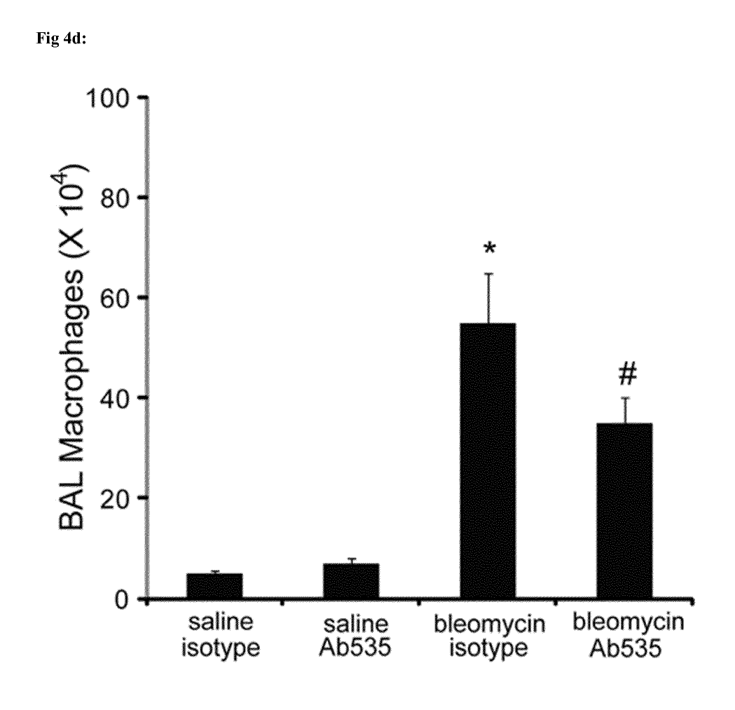

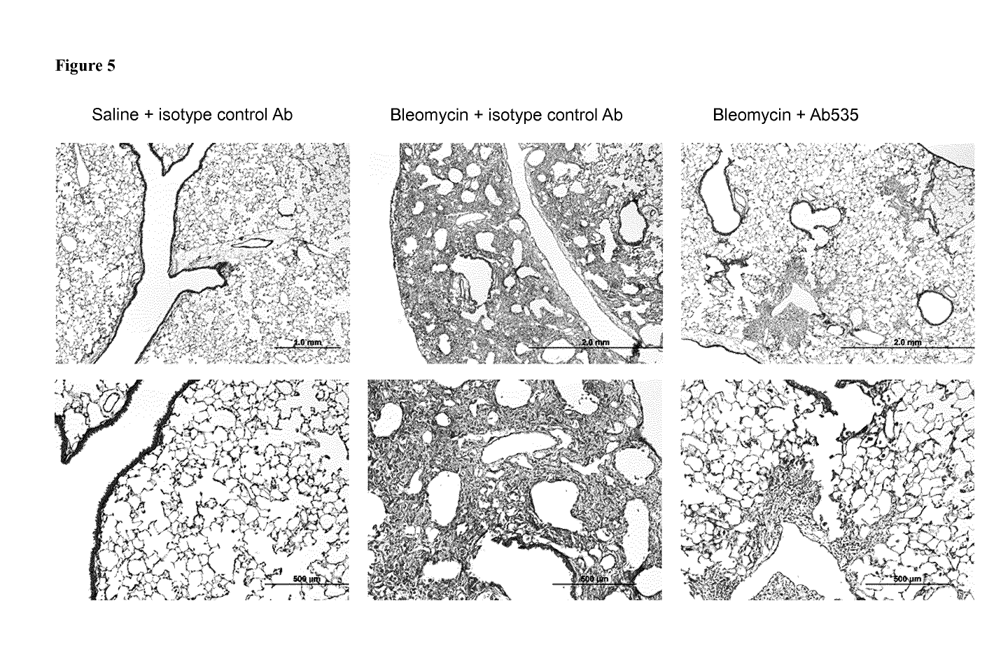

FIG. 4a shows that treatment of bleomycin-induced lung fibrosis with Ab535 reduced the amount of collagen in BAL fluid compared to treatment with the isotype control.
FIG. 4b shows that treatment of bleomycin-induced lung fibrosis with Ab535 reduced the Ashcroft score of the samples compared to treatment with the isotype control demonstrating that the mice treated with Ab535 had improved fibrotic pathology.
FIG. 4c shows that treatment of bleomycin-induced lung fibrosis with Ab535 reduced the amount of albumin in BAL fluid compared to treatment with the isotype control demonstrating that the vascular permeability and pulmonary barrier integrity of the mice treated with Ab535 was improved.
FIG. 4d shows that treatment of bleomycin-induced lung fibrosis with Ab535 reduced the number of macrophages in the BAL fluid.
FIG. 5 shows representative images of the histopathological analysis of lungs from saline control, bleomycin plus isotype control and bleomycin plus Ab 535 treated animals. Animals treated with Ab 535 had a greatly reduced fibrosis of the lungs compared to the isotype control, bleomycin treated animals.

### EXAMPLE 4

### Effect of Anti-CSF-1R Antibody in an Adenosine Deaminase-Deficient Mouse Model of Pulmonary Fibrosis

Adenosine is a potent signalling nucleoside, the levels of which increase when cells suffer stress or are damaged and a wide variety of responses are produced when adenosine engages its specific G protein coupled receptors. Adenosine deaminase (ADA) is a purine catabolism enzyme that converts adenosine to inosine. ADA knockout mice have been generated and shown to have increased levels of adenosine in serum as well as in tissues such as kidney, liver and lung (Blackburn, M R et al, 1998, J Biol Chem, 273(9): 5093-5100). These mice exhibit features of chronic lung disease such as alveolar destruction, airway inflammation and excessive mucus production which are associated with increased levels of adenosine in the lung (Blackburn M R et al, 2000, J Exp Med, 192: 159-70). The effects are such that the mice die by three weeks of age due to respiratory distress. Administration of exogenous ADA using a low-dose regimen reduces adenosine levels and extends the life-span of these mice allowing a model of pulmonary fibrosis to be developed (Chunn J L et al, 2005, J Immunol 175: 1937-46, Pedrosa M et al, 2011, PLoS One, 6(7): e22667). In this model, chronic elevation of adenosine levels is associated with an increase in pro-fibrotic mediators including TGFβ-1 in the lungs, increased collagen deposition in lung tissue and increased fibrotic lung pathology. To investigate the effect of molecules with anti-fibrotic potential, ADA-deficient mice are maintained on a low dose exogenous ADA regimen for several weeks, the ADA treatment is then stopped and the potential anti-fibrotic agent administered.

The effect of the anti-CSF-1R antibody Ab535 was investigated in the ADA knockout mouse model of pulmonary fibrosis. In this model, the enzyme deficient mice were maintained on ADA enzyme therapy from day 1 to day 21 post-birth. An ADA-polyethylene glycol (PEG) conjugate was prepared (Young H W et al, 2004, J Immunol 173: 1380-89) and intramuscular injections administered on postnatal days 1, 5, 9, 13 and 17 (0.625, 1.25, 2.5, 2.5 and 2.5 units respectively), followed by 5 units injected intraperitoneally on day 21. No further enzyme was administered after day 21. Ab535 was dosed subcutaneously at 30 mg/kg, in a volume of 100 μl three times/week from day 25 (post-natally) until the animals were sacrificed on day 42.

Animals were sacrificed at day 42 and readouts included histopathological analysis of the lungs, BAL fluid cellularity and quantitation of soluble collagen in BAL fluid. Histopathological analysis was conducted to score lung damage using a modified Ashcroft scoring system to determine the severity of lung fibrosis (Hubner et al, 2008, Biotechniques 44: 507-17). Excised lungs were inflated with 10% formalin to 25 cm pressure and processed through a series of alcohols and xylene, embedded in paraffin and tissue sections de-paraffinised prior to processing and staining with Masson's Trichrome.

The amount of soluble collagen in BAL fluid was also assessed using a commercially available Sircol assay kit following the manufacturer's instructions.

The effect on the macrophage population in the BAL fluid was determined using cytospin preparations. Aliquots of BAL cells were spun onto microscope slides, stained with Diff-Quick and macrophages counted.

It was found that therapeutic treatment with anti-CSF-1R antibody Ab535 significantly reduced lung fibrosis in ADA-deficient mice. The treated mice had reduced collagen production, improved fibrotic pathology and improved pulmonary barrier function and protection.

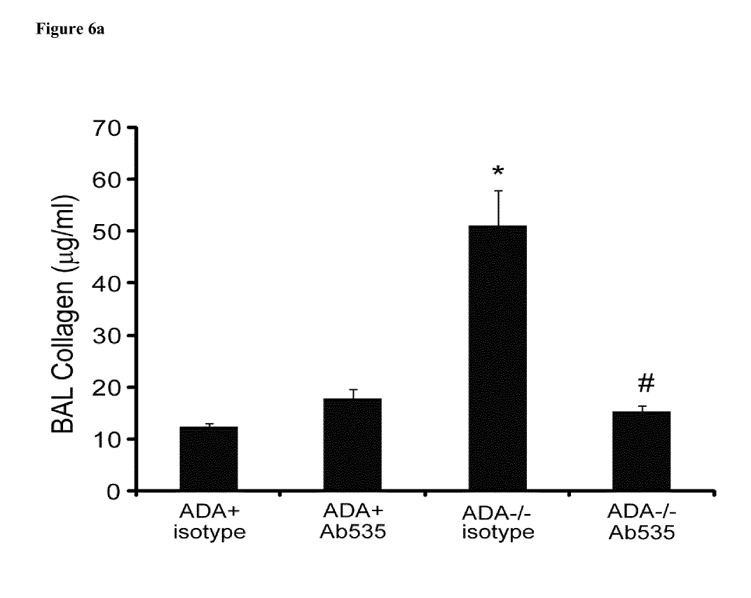

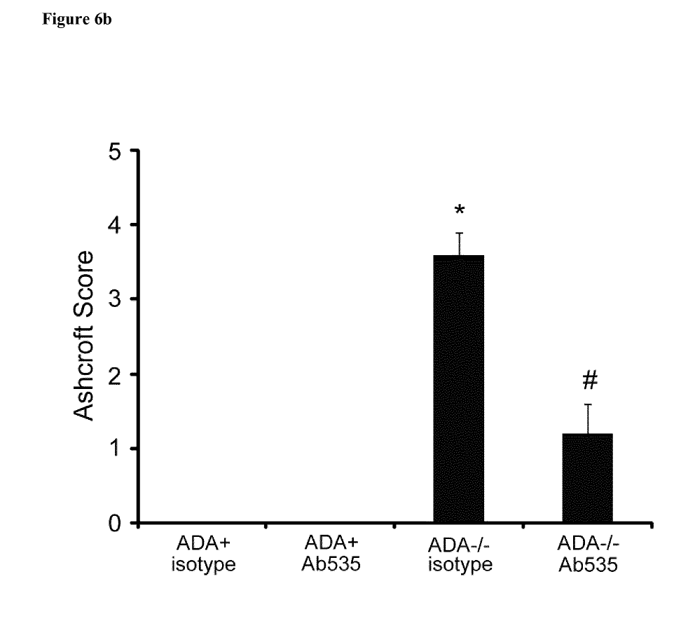

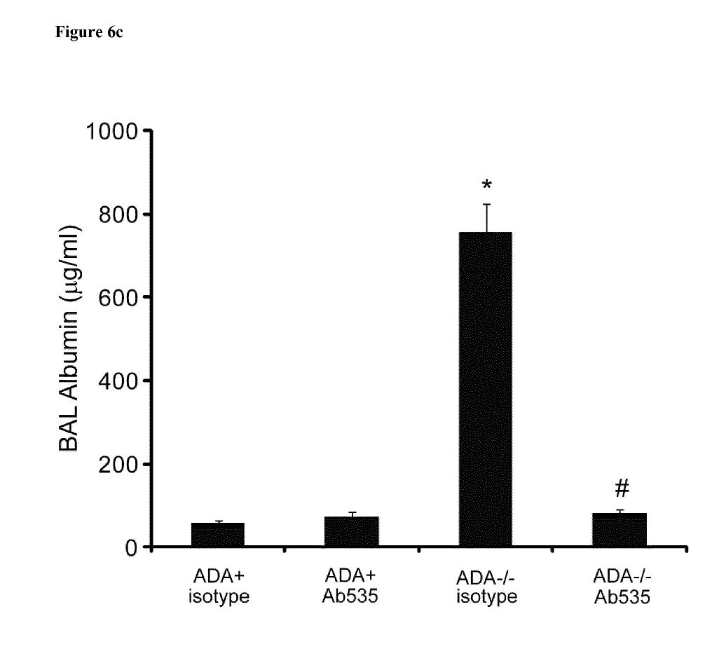

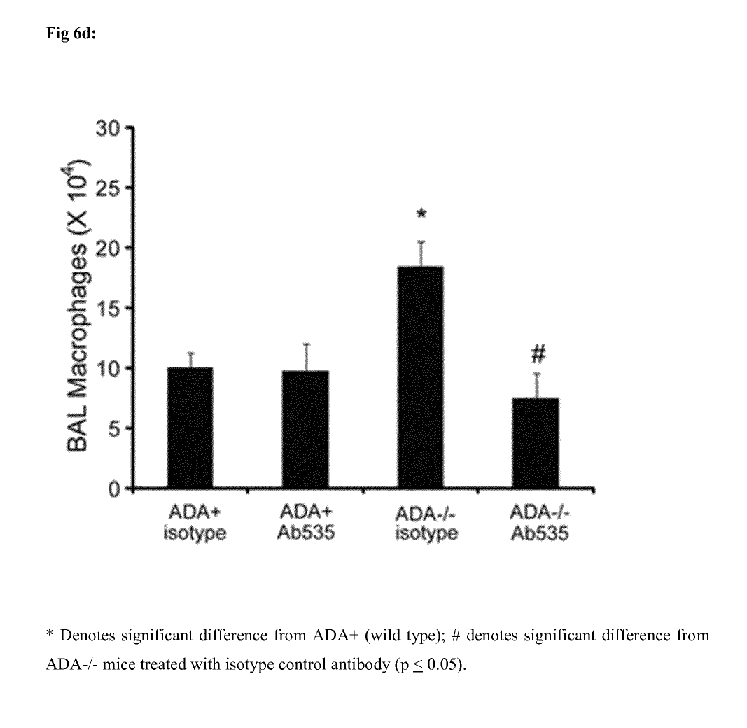

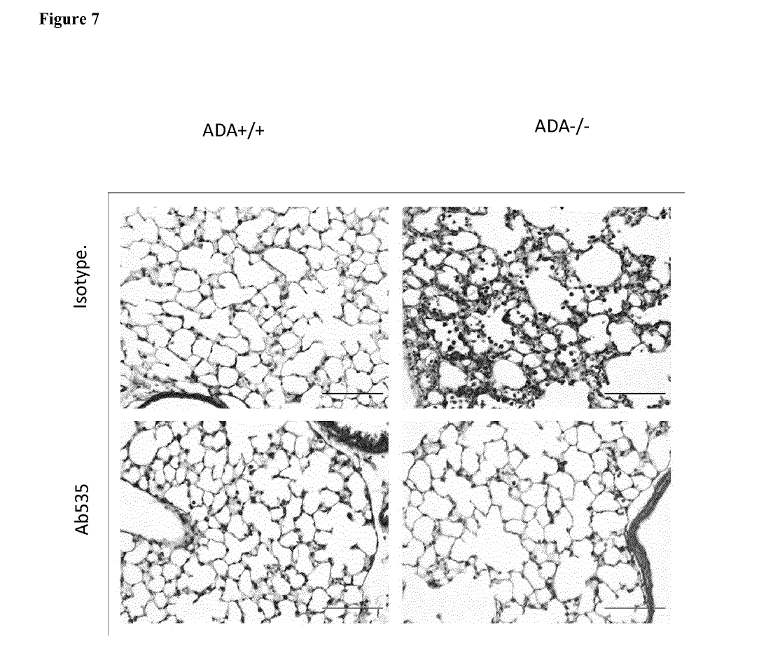

FIG. 6a shows that treatment of ADA-deficient mice with induced lung fibrosis with Ab535 reduced BAL fluid collagen concentration compared to treatment with the isotype control.
FIG. 6b shows that treatment of ADA-deficient mice with induced lung fibrosis with Ab535 reduced the Ashcroft score of the samples compared to treatment with the isotype control demonstrating that the mice treated with Ab535 had improved fibrotic pathology.
FIG. 6c shows that treatment of ADA-deficient mice with induced lung fibrosis with Ab535 reduced the concentration of albumin in the BAL fluid compared to treatment with the isotype control demonstrating that the vascular permeability and pulmonary barrier integrity of the mice treated with Ab535 was improved.
FIG. 6d shows that treatment of ADA-deficient mice with induced lung fibrosis, with Ab535, reduced the number of macrophages in BAL fluid.
FIG. 7 shows representative images of the histopathological analysis of lung tissue from ADA knockout mice and wild type mice either treated with Ab535 or an isotype control antibody (mouse IgG1). Lung tissue sections were stained with Masson's trichrome. Treatment of ADA knockout mice with Ab535 greatly reduced the amount of fibrotic tissue and collagen deposition.

Accordingly, it has been shown in two mouse models of lung fibrosis that treatment with an anti-CSF-1R antibody can effectively treat fibrotic disease.

1-35. (canceled)

36. An inhibitor of CSF-1R activity for the treatment and/or prophylaxis of fibrotic disease.

37. The inhibitor of CSF-1R activity according to claim 36, wherein the inhibitor is a small chemical entity (NCE).

38. The inhibitor according to claim 36, wherein the inhibitor is a nucleic acid.

39. The inhibitor according to claim 36, wherein the inhibitor is an antibody or functionally active fragment or derivative thereof.

40. The inhibitor according to claim 39, wherein the antibody or fragment thereof is monoclonal, polyclonal, chimeric, humanised or bispecific.

41. The inhibitor according to claim 39, wherein the antibody fragment is a Fab, Fab′, F(ab′)₂, scFv or an epitope-binding fragment thereof.

42. The inhibitor according to claim 39, wherein the antibody or fragment thereof is conjugated to one or more effector molecule(s).

43. The inhibitor according to claim 39, wherein the antibody or fragment thereof binds to CSF-1R.

44. The inhibitor according to claim 36, wherein the fibrotic disease is:
a) idiopathic pulmonary fibrosis (IPF); or
b) scleroderma.

45. A method for the treatment and/or prophylaxis of fibrotic disease comprising administering a therapeutically effective amount of an inhibitor of CSF-1R activity.

46. The method according to claim 45, wherein the inhibitor is a small chemical entity (NCE).

47. The method according to claim 45, wherein the inhibitor is a nucleic acid.

48. The method according to claim 45, wherein the inhibitor is an antibody or functionally active fragment or derivative thereof.

49. The method according to claim 48, wherein the antibody or fragment thereof is monoclonal, polyclonal, chimeric, humanised or bispecific.

50. The method according to claim 48, wherein the antibody fragment is a Fab, Fab′, F(ab′)₂, scFv or epitope binding fragment thereof.

51. The method according to claim 48, wherein the antibody or fragment thereof is conjugated to one or more effector molecule(s).

52. The method according to claim 48, wherein the antibody binds to CSF-1R.

53. The method according to claim 45, wherein the fibrotic disease is:
a) idiopathic pulmonary fibrosis (IPF); or
b) scleroderma; or
c) renal fibrosis.

54. The method according claim 45, wherein the inhibitor of CSF-1R activity is administered in combination with one or more other therapeutically active compounds.

55. The method according to claim 54 in which the other therapeutically active compound is another anti-fibrotic therapeutic agent.
